SEQUENCE LISTING (6 sequences)

SEQ ID NO: 1 - DNA, 13394 bp, metapneumovirus
gcgaaaaaaacgcgtataaattagattacaaaaaaatatgggacaagtgaaaatgtctcttcaagggattcacctgagtgatctatcatacaagcatgctatattaaaagagtctcagtacacaataaaaagagatgtgggtacaacaactgcagtgacaccctcatcattgcaacaagaaataacgctgttgtgtggagaaattctgtatgctaaacatgctgattacaaatatgctgcagaaataggaatacaatatattagcacagctttaggatcagagagagtgcagcagattctgaggaactcaggcagtgaagtccaagtggtcttaaccagaacgtactctctggggaaagttaaaaacaataaaggagaagatttacagatgttagacatacacggggtagagaagagctgggtagaagagatagacaaagaagcaaggaaaacaatggcaaccttgcttaaggaatcatcaggtaatatcccacaaaatcagaggccctcagcaccagacacacccataatcttattatgtgtaggtgcattaatatttactaagctagcatcaaccatagaagtgggactagagaccacagtcagaagggctaaccgtgtactaagtgatgcactcaagagataccctagaatggacatcccaaaaattgccagatccttctatgacttatttgaacaaaaagtgtatcacagaagtttgttcattgagtatggcaaagcattaggctcatcatctacaggcagcaaagcagaaagtctatttgttaatatattcatgcaagcttatggagccggtcaaacaatgctaaggtggggggtcattgccaggtcatccaacaatataatgttaggacatgtatctgtccaagctgagttaaaacaggtcacagaagtctatgacttggtgcgagaaatgggccctgaatctggacttctacatttaaggcaaagcccaaaagctggactgttatcactagccaactgtcccaactttgcaagtgttgttctcggaaatgcctcaggcttaggcataatcggtatgtatcgtgggagagtaccaaacacagaattattttcagcagcagaaagttatgccaaaagtttgaaagagagcaataaaatcaatttctcttcattaggacttacagatgaagagaaagaggctgcagaacatttcttaaatgtgagtgacgacagtcaaaatgattatgagtaattaaaaaagtgggacaagtcaaaatgtctttccctgaaggaaaagatattcttttcatgggtaatgaagcagcaaaattagcagaagctttccagaaatcattaaggaaaccaagtcataaaagatctcaatctattataggagaaaaagtgaacactgtatcagaaacattggaattacctactatcagtagacctgcaaaaccaaccatactgtcagaaccaaagttagcatggactgataaaggtggggcaatcaaaactgaaataaagcaagcaatcaaagtcatggatcctattgaggaagaagagtctactgagaagaaggtgctgccctccagtgatgggaaaacccctgcagaaaagaaactgaaaccatcaactaacaccaaaaagaaagtttcgtttacaccaaatgaaccaggaaaatatacaaagttggaaaaagatgctctagatttgctctcagataatgaagaagaagatgcagaatcttcaatcttaacctttgaagaaagagatacttcatcgttaagcattgaggccagattggaatcaatagaggagaaattaagcatgatattagggctattaagaacactcaacattgctacagcaggacccacagcagcaagagatgggatcagagatgcaatgattggcgtaagagaggaattaatagcagacataataaaggaagccaaagggaaagcagcagaaatgatggaagaggaaatgagtcaacgatcaaaaataggaaacggtagtgtaaaactaacagagaaagcaaaagagcttaacaaaattgttgaagatgaaagcacaagtggagaatctgaagaagaagaagaaccaaaagacatacaagacaatagtcaagaagatgacatttaccagttaattatgtagtttaataaaaataaacaatgggacaagtaaaaatggagtcctacctagtagacacttatcaaggcattccttacacagcagctgttcaagttgatctaatagaaaaggacctgttacctgcaagcctaacaatatggttccctttgtttcaggccaacacaccaccagcagtgctgctcgatcagttgaaaaccctaacaataaccactctgtatgctgcatcacaaaatggtccaatactcaaagtgaatgcatcagcccaaggtgcagcaatgtctgtacttcccaaaaaatttgaagtcaatgcgactgtagcactcgatgaatatagcaaattggaatttgacaaactcacagtctgtgaagtaaaaacagtttacttaacaaccatgaaaccatacgggatggtatcaaaatttgtgagctcagccaaatcagttggcaaaaaaacacatgatctaatcgcactgtgtgattttatggatctagaaaagaacacacctgttacaataccagcattcatcaaatcagtttcaatcaaagagagtgagtcagctactgttgaagctgctataagcagtgaagcagaccaagctctaacacaggccaaaattgcaccttatgcgggattgattatgatcatgactatgaacaatcccaaaggcatattcaaaaagcttggagctgggactcaagttatagtagaactaggagcatatgtccaggctgaaagcataagtaaaatatgcaagacttggagccatcaagggacaagatatgtgttgaagtccagataacagccaagcaccttggccaagagctactaactctatctcatagattataaagtcaccattctagttatataaaaatcaagttagaacaagaattaaatcaatcaagaatgggacaaataaaaatgtcttggaaagtggtgatcattttttcattgttaataacacctcaacacggtcttaaagagagctatttagaagagtcatgtagcactataactgaaggatatctcagtgttctgaggacaggttggtataccaacgtttttacactggaggtaggtgatgtagagaaccttacatgtgctgatggacctagcttaataaaaacagaattagacctgaccaaaagtgcactaagagaactcagaacagtttctgctgatcaactggcaagagaggagcaaattgagaatcccagacaatctagatttgttctaggagcaatagcactcggtgttgcaacagcagctgcagttacagcaggtgttgcaattgccaaaaccatccggcttgaaagtgaagtaacagcaattaagaatgccctcaaaaagaccaatgaagcagtatctacattggggaatggagttcgagtgttggcaactgcagtgagggagctggaagattttgtgagcaagaatctaacacgtgcaatcaacaaaaacaagtgcgacattgctgacctgaaaatggccgttagcttcagtcaattcaacagaaggtttctaaatgttgtgcggcaattttcagacaatgctggaataacaccagcaatatccttggacttaatgacagatgctgaactagccagagctgtttccaacatgccaacatctgcaggacaaataaaactgatgttggagaaccgtgcaatggtaagaagaaaggggttcggaatcctgataggagtttacggaagctccgtaatttacatggtgcaactgccaatctttggagttatagacacgccttgctggatagtaaaagcggccccttcttgctcagaaaaaaagggaaactatgcttgccttttaagagaagatcaaggatggtattgtcagaatgcagggtcaactgtttactacccaaatgaaaaagactgcgaaacaagaggagaccatgtcttttgcgacacagcagcaggaatcaatgttgctgagcagtcaaaggagtgcaacatcaacatatccactactaattacccatgcaaagttagcacaggaagacaccctatcagtatggttgcactgtctcctcttggggctttggttgcttgctacaagggagtgagctgttccattggcagcaacagagtagggatcatcaagcaactgaacaaaggctgctcttatataaccaaccaagacgcagacacagtgacaatagacaacactgtataccagctaagcaaagttgagggcgaacagcatgttataaaaggaaggccagtgtcaagcagctttgatccagtcaaatttcctgaagatcaattcaatgttgcacttgaccaagttttcgaaagcattgagaacagtcaggccttggtggatcaatcaaacagaatcctaagcagtgcagagaaaggaaacactggcttcatcattgtaataattctaattgctgtccttggctctaccatgatcctagtgagtgtttttatcataataaagaaaacaaagaaacccacaggagcacctccagagctgagtggtgtcacaaacaatggcttcataccacataattagttaattaaaaataaagtaaattaaattaaaataaaataaaattaaaattaaaataaaataaaaataaaaatttgggacaaatcataatgtctcgcaaggctccatgcaaatatgaagtgcggggcaaatgcaatagaggaagtgagtgcaagtttaaccacaattactggagttggccagatagatacttactaataagatcaaattatttattaaatcaacttttaaggaacactgatagagctgatggcttatcaataatatcaggagcaggcagagaagataggacacaagattttgtcctaggttccaccaatgtggttcaaggttatattgatgataaccaaagcataacaaaagctgcagcctgttacagtctacataatataatcaaacaactacaagaagttgaagttaggcaggctagagataacaaaccatctgacagcaaacatgtggcacttcacaacttagtcctatcttatatggagatgagcaaaattcctgcatctttaatcaacaatctcaaaagactgccgagagagaaactgaaaaaattagcaaagcttataattgacttatcagcaggtgctgaaaatgactcttcatatgccttgcaagacagtgaaagcactaatcaagtgcagtgagcatggtcctgttttcattactatagaggttgattacatgatatggactcataaggacttaaaagaagctttatctaatgggatagtgaagtctcatactaacatttacaattgttatttagaaaacatagaaattatatatgtcaaggcttacttaagttagtaaaaacacatcagagtgggataaatgacaatgataacattagatgtcattaaaagtgatgggtcttcaaaaacatgtactcacctcaaaaaattaattaaagaccactctggtaaagtgcttattgtacttaagttaatattagctttactaacatttctcacagtgacaatcaccatcaattatataaaagtagaaaacaatctgcaaatatgtcagtcaaaaactgaatcagacaaaaaggactcatcatcaaataccacatcagtcacaaccaagactactctaaatcatgatataacacagtattttaaaagtttgattcaaaggtatacaaactctgcaataaacagagacacatgctggaaaataagcagaaatcaatgcacaaacataacaacatacaaatttttatgttttaaatctgaagaaacaaaaaccaacaattgtgataaactgacagatttatgcagaaacaaaccaaaaccagctgttgaagtgtatcacatagtagaatgccattgtatatacacagttaaatggaagtgctatcattacccaatagatgaaacccaatcctaaataacactagattaggatccatccaagtctgttagttcaacaatttagttatttaaaaatattttgaaaacaagtaagtttctatgatacttcataataataagtaataattaattgcttaatcatcatcacaacattattcgaaaccataactattcaatttaagaagtaaaaacaataatatgggacaagtagttatggaggtgaaagtggagaacattcgaacaatagatatgctcaaagcaagagtgaaaaatcgtgtggcacgcagcaaatgctttaaaaatgcctctttgatcctaataggaataactacattgagtatagccctcaatatctatctaatcataaactatacaatgcaagaaaacacatccgaatcagaacatcacaccagctcatcacccatggaatccagcagggaaactccaacagtccctatggacaactcagacaccaatccaggctcacagtatccaactcaacagtccacagaaggctccacactctactttgcagcctcagcaagctcaccagagacagaaccaacatcaacaccagacacaacaagccgcccgcccttcgtcgacacacacacaacactaccaagtgcaagcagaacaaggacaagtccggcagtccacacaaaaaacaatccaaggacaagccccagaacacattccccaccatgggcaatgacaaggacggtccgtggaaccaccactctccgcacaagcagcacaagaaaaagaccgtccacagcatcagtccaacctgacagcagcgcaacaacccacaaacacgaagaagcaagcccagtgagcccgcaaacatctgcgagcacagcaagaccacaaaggaagggcatggaggccagcacatcaacaacatacaaccaaactagttaacaaaaaatacaaaataactctaagataaaccatatagacaccaacaattgagaagccaaaaggcaattcacaatctctccaaaaaggcaacaacaccatattagctccgcttaaatctccctggaaaaaacactcgcccatataccaactataccacaaccatcccaagaaaaaaagctgggtaaaacaacacccaagagacaaataacaatggatcctcttaatgaatccactgttaatgtctatcttcccgactcatatcttaaaggagtgatttcttttagtgagactaatgcaattggttcatgtctcttaaaaagaccttacctaaaaaatgacaacactgcaaaagttgccatagagaatcctgttatcgagcatgttagactcaaaaatgcaatcaattctaagatgaaaatatcagattacaggatagtagagccagtaaacatgcaacatgaaattatgaagaatgtacacagttgtgagctcacattattaaaacagtttttaacaaggagtaaaaatattagcactcttaaattaaatatgatatgtgattggctgcagttaaagtctacatcagatgatacctcaatcttaagttttatagatgtagaatttatacctagctgggtaagcaattggtttagtaattggtacaatctcaacaagttgattctggaattcaggaaagaagaagtaataagaactggttcaatcttgtgtaggtcattgggtaaattagtttttgttgtatcatcatacggatgtatagtcaagagcaacaaaagcaaaagagtgagcttcttcacatacaatcaactgttaacatggaaagatgtgatgttaagtagattcaatgcaaatttctgtatatgggtaagcaacagtctgaatgaaaatcaagaagggttagggttgagaagtaatctgcaaggcatattaactaataagctatatgaaactgtagattatatgcttagtttgtgttgcaatgaaggtttctcacttgtgaaagagtttgagggttttattatgagtgaaatccttaggattactgaacatgctcaattcagtactagatttagaaatactttattaaatggattaactgatcaattgacaaaattaaaaaataaaaacagactcagagttcatggtaccgtgttagaaaataatgattatccaatgtatgaagttgtacttaaattattaggagatactttgagatgtattaaattattaatcaataaaaacttagagaatgctgctgaattatactatatatttagaatattcggtcacccaatggtagatgaaagagatgcaatggatgctgtcaaattaaacaatgaaatcacaaaaatcctcaggttggagagcttgacagaactaagaggggcattcatattaaggattatcaaaggatttgtagacaacaacaaaagatggccgaaaattaaaaacttaaaagtgcttagtaaaagatggactatgtacttcaaagcaaaaagttaccctagtcaacttgaattaagtgaacaagattttttagagcttgctgcaatacagtttgaacaagagttttctgttcctgaaaaaaccaaccttgagatggtattaaatgataaagctatatcacctcctaaaagattaatttggtctgtgtacccaaaaaattacttacctgagacaataaaaaatcgatatctagaagagactttcaatgcaagtgatagtctcaaaacaagaagagtactagagtactatttgaaagataataaattcgaccaaaaagaacttaaaagttatgtggttaaacaagaatatttaaatgataaggatcatattgtctcgctaactggaaaagaaagagaattaagtgtaggtagaatgtttgctatgcaaccaggaaaacagcgacaaatacaaatattggctgaaaaattgttagctgataatattgtaccctttttcccagaaactttaacaaagtatggtgatctagatcttcagagaataatggaaatcaaatcagaactttcttctattaaaaccagaagaaatgatagttataataattacattgcaagagcatccatagtaacagatttaagtaagttcaaccaagcctttaggtatgaaactacagcgatctgtgcggatgtagcagatgaactacatggaacacaaagcctattctgttggttacatcttatcgttcctatgactacaatgatatgtgcctatagacatgcaccaccagaaacaaaaggtgaatatgatatagataagatagaagagcaaagtggtttatatagatatcatatgggtggtattgaaggatggtgtcaaaaactctggacaatggaagctatatctttattagatgttgtatctgtaaagacacgatgtcaaatgacatctttattaaacggtgacaaccaatcaatagatgtaagtaaaccagttaagttatctgagggtttagatgaagtgaaagcagattatagcttggctgtaaaaatgctaaaagaaataagagatgcatacagaaatataggccataaacttaaagaaggggaaacatatatatcaagagatcttcagtttataagtaaggtgattcaatctgaaggagtaatgcatcctacccctataaaaaagatcttaagagtgggaccatggataaacacaatattagatgacattaaaaccagtgcagagtcaatagggagtctatgtcaggaattagaatttaggggggaaagcataatagttagtctgatattaaggaatttttggctgtataatttatacatgcatgaatcaaagcaacaccccctagcagggaagcagttattcaaacaactaaataaaacattaacatcagtgcagagattttttgaaattaaaaaggaaaatgaagtagtagatctatggatgaacataccaatgcagtttggaggaggagatccagtagtcttctatagatctttctatagaaggacccctgattttttaactgaagcaatcagtcatgtagatattctgttaaaaatatcagccaacataagaaatgaagcgaaaataagtttcttcaaagccttactgtcaatagaaaaaaatgaacgtgctacactgacaacactaatgagagaccctcaagctgtgggctcagagcgacaagcaaaagtaacaagtgatatcaatagaacagcagttaccagcatcttaagtctttctccaaatcaacttttcagcgatagtgctatacactacagtagaaatgaagaagaggtcggaatcattgctgacaacataacacctgtttatcctcatggactgagagttttgtatgaatcattaccttttcataaagctgaaaaagttgtaaatatgatatcaggaacaaaatccataaccaacttattacagagaacatctgctattaatggtgaagatattgacagagctgtatccatgatgctggagaacctaggattattatctagaatattgtcagtagttgttgatagtatagaaattccaaccaaatctaatggtaggctgatatgttgtcagatatctagaaccctaagggagacatcatggaataatatggaaatagttggagtaacatcccctagcatcactacatgcatggatgtcatatatgcaactagctctcatttgaaagggataatcattgaaaagttcagcactgacagaactacaagaggtcaaagaggtccaaagagcccttgggtaggatcgagcacgcaagagaaaaaattagttcctgtttataacagacaaattctttcaaaacaacaaagagaacagctagaagcaattggaaaaatgagatgggtatataaagggacaccaggtttaagacgattactcaataagatttgtcttggaagtttaggcattagttacaaatgtgtaaaacctttattacctaggtttatgagtgtaaatttcctacacaggttatctgtcagtagtagacctatggaattcccagcatcagttccagcttatagaacaacaaattaccattttgacactagtcctattaatcaagcactaagtgaaagatttgggaatgaagatattaatttggtcttccaaaatgcaatcagctgtggaattagcataatgagtgtagtagaacaattaactggtaggagtccaaaacagttagttttaataccccaattagaagaaatagacattatgccaccaccagtgtttcaagggaaattcaattataagctagtagataagataacttctgaccaacatatcttcagtccagacaaaatagatatgttaacactggggaaaatgctcatgccaactataaaaggtcagaaaacagatcagttcttgaacaagagagagaattatttccatgggaacaatcttattgagtctttgtcagcagcgttagcatgtcattggtgtgggatattaacagagcaatgtatagaaaataatattttcaagaaagactggggtgacgggttcatatcggatcatgcttttatggacttcaaaatattcctatgtgtctttaaaactaaacttttatgtagttggggatcccaagggaaaaacattaaagatgaagatatagtagatgaatcgatagataaactgttaaggattgataatactttttggagaatgttcagcaaggttatgtttgaatcaaaggttaagaaaaggataatgttatatgatgtaaaatttctatcattagtaggttatatagggtttaagaattggtttatagaacagttgagatcagctgagttgcatgaggtaccttggattgtcaatgccgaaggtgatctggttgagatcaagtcaattaaaatctatttgcaactgatagagcagagtttatttttaagaataactgttttgaactatacagatatggcacatgctctcacaagattaatcagaaagaagttgatgtgtgataatgcactattaacttcaattccatccccaatggttaacttaactcaagttattgatcctacagaacaattagcttatttccctaagataacatttgaaaggctaaaaaattatgacactagttcaaattatgctaaaggaaagctaacaaggaattacatgatactgttgccatggcaacatgttaatagatataactttgtctttagttctactggatgtaaagttagtctaaaaacatgcattggaaaacttatgaaagatctaaaccctaaagttctgtactttattggagaaggggcaggaaattggatggccagaacagcatgtgaatatcctgacatcaaatttgtatacagaagtttaaaagatgaccttgatcatcattatcctttggaataccagagagtgataggagaattaagcaggataatagatagtggtgaagggctttcaatggaaacaacagatgcaactcaaaaaactcattgggatttgatacacagagtaagcaaagatgctttattaataactttatgtgatgcagaatttaaggacagagatgatttttttaagatggtaattctatggaggaaacatgtattatcatgcagaatttgcactacctatgggacagacctctatttattcgcaaagtatcatgctaaagactgcaatataaaattacctttttttgtgagatcagttgccacctttattatgcaaggtagtaaactgtcaggctcggaatgctacatactcttaacactaggccaccacaacaatttaccttgtcatggagaaatacaaaattctaagatgaaaatagcagcgtgtaatgatttttatgctgcaaaaaaacttgacaataaatcaattgaagccaactgtaaatcacttttatcagggctaagaataccgataaataagaaggaattaaatagacagagaaggttattaacactacaaagcaaccattcttctgtagcaacagttggaggtagcaaggtcatagagtctaaatggttaacaaacaaggcaaacacaataattgattggttagaacatattttaaattctccaaaaggtgaattaaattatgatttttttgaagcattagaaaatacttaccctaatatgattaaactaatagataatctagggaatgcagagattaaaaaactgatcaaagtaactggatatatgcttgtaagtaaaaaatgaaaaatgataaaaatgataaaataggtgacaacttcatactattccaaagtaatcatttgattatgcaattatgtaatagttaattaaaaactaaaaatcaaaagttaaaaactaataactgtcattaagtttattaaaaataagaaattataattggatgtatacggtttttttgccgt

SEQ ID NO: 2 - DNA, 12645 bp, metapneumovirus
gcgaaaaaaacgcgtataaattagattacaaaaaaatatgggacaagtgaaaatgtctcttcaagggattcacctgagtgatctatcatacaagcatgctatattaaaagagtctcagtacacaataaaaagagatgtgggtacaacaactgcagtgacaccctcatcattgcaacaagaaataacgctgttgtgtggagaaattctgtatgctaaacatgctgattacaaatatgctgcagaaataggaatacaatatattagcacagctttaggatcagagagagtgcagcagattctgaggaactcaggcagtgaagtccaagtggtcttaaccagaacgtactctctggggaaagttaaaaacaataaaggagaagatttacagatgttagacatacacggggtagagaagagctgggtagaagagatagacaaagaagcaaggaaaacaatggcaaccttgcttaaggaatcatcaggtaatatcccacaaaatcagaggccctcagcaccagacacacccataatcttattatgtgtaggtgcattaatatttactaagctagcatcaaccatagaagtgggactagagaccacagtcagaagggctaaccgtgtactaagtgatgcactcaagagataccctagaatggacatcccaaaaattgccagatccttctatgacttatttgaacaaaaagtgtatcacagaagtttgttcattgagtatggcaaagcattaggctcatcatctacaggcagcaaagcagaaagtctatttgttaatatattcatgcaagcttatggagccggtcaaacaatgctaaggtggggggtcattgccaggtcatccaacaatataatgttaggacatgtatctgtccaagctgagttaaaacaggtcacagaagtctatgacttggtgcgagaaatgggccctgaatctggacttctacatttaaggcaaagcccaaaagctggactgttatcactagccaactgtcccaactttgcaagtgttgttctcggaaatgcctcaggcttaggcataatcggtatgtatcgtgggagagtaccaaacacagaattattttcagcagcagaaagttatgccaaaagtttgaaagagagcaataaaatcaatttctcttcattaggacttacagatgaagagaaagaggctgcagaacatttcttaaatgtgagtgacgacagtcaaaatgattatgagtaattaaaaaagtgggacaagtcaaaatgtctttccctgaaggaaaagatattcttttcatgggtaatgaagcagcaaaattagcagaagctttccagaaatcattaaggaaaccaagtcataaaagatctcaatctattataggagaaaaagtgaacactgtatcagaaacattggaattacctactatcagtagacctgcaaaaccaaccatactgtcagaaccaaagttagcatggactgataaaggtggggcaatcaaaactgaaataaagcaagcaatcaaagtcatggatcctattgaggaagaagagtctactgagaagaaggtgctgccctccagtgatgggaaaacccctgcagaaaagaaactgaaaccatcaactaacaccaaaaagaaagtttcgtttacaccaaatgaaccaggaaaatatacaaagttggaaaaagatgctctagatttgctctcagataatgaagaagaagatgcagaatcttcaatcttaacctttgaagaaagagatacttcatcgttaagcattgaggccagattggaatcaatagaggagaaattaagcatgatattagggctattaagaacactcaacattgctacagcaggacccacagcagcaagagatgggatcagagatgcaatgattggcgtaagagaggaattaatagcagacataataaaggaagccaaagggaaagcagcagaaatgatggaagaggaaatgagtcaacgatcaaaaataggaaacggtagtgtaaaactaacagagaaagcaaaagagcttaacaaaattgttgaagatgaaagcacaagtggagaatctgaagaagaagaagaaccaaaagacatacaagacaatagtcaagaagatgacatttaccagttaattatgtagtttaataaaaataaacaatgggacaagtaaaaatggagtcctacctagtagacacttatcaaggcattccttacacagcagctgttcaagttgatctaatagaaaaggacctgttacctgcaagcctaacaatatggttccctttgtttcaggccaacacaccaccagcagtgctgctcgatcagttgaaaaccctaacaataaccactctgtatgctgcatcacaaaatggtccaatactcaaagtgaatgcatcagcccaaggtgcagcaatgtctgtacttcccaaaaaatttgaagtcaatgcgactgtagcactcgatgaatatagcaaattggaatttgacaaactcacagtctgtgaagtaaaaacagtttacttaacaaccatgaaaccatacgggatggtatcaaaatttgtgagctcagccaaatcagttggcaaaaaaacacatgatctaatcgcactgtgtgattttatggatctagaaaagaacacacctgttacaataccagcattcatcaaatcagtttcaatcaaagagagtgagtcagctactgttgaagctgctataagcagtgaagcagaccaagctctaacacaggccaaaattgcaccttatgcgggattgattatgatcatgactatgaacaatcccaaaggcatattcaaaaagcttggagctgggactcaagttatagtagaactaggagcatatgtccaggctgaaagcataagtaaaatatgcaagacttggagccatcaagggacaagatatgtgttgaagtccagataacagccaagcaccttggccaagagctactaactctatctcatagattataaagtcaccattctagttatataaaaatcaagttagaacaagaattaaatcaatcaagaatgggacaaataaaaatgtcttggaaagtggtgatcattttttcattgttaataacacctcaacacggtcttaaagagagctatttagaagagtcatgtagcactataactgaaggatatctcagtgttctgaggacaggttggtataccaacgtttttacactggaggtaggtgatgtagagaaccttacatgtgctgatggacctagcttaataaaaacagaattagacctgaccaaaagtgcactaagagaactcagaacagtttctgctgatcaactggcaagagaggagcaaattgagaatcccagacaatctagatttgttctaggagcaatagcactcggtgttgcaacagcagctgcagttacagcaggtgttgcaattgccaaaaccatccggcttgaaagtgaagtaacagcaattaagaatgccctcaaaaagaccaatgaagcagtatctacattggggaatggagttcgagtgttggcaactgcagtgagggagctggaagattttgtgagcaagaatctaacacgtgcaatcaacaaaaacaagtgcgacattgctgacctgaaaatggccgttagcttcagtcaattcaacagaaggtttctaaatgttgtgcggcaattttcagacaatgctggaataacaccagcaatatccttggacttaatgacagatgctgaactagccagagctgtttccaacatgccaacatctgcaggacaaataaaactgatgttggagaaccgtgcaatggtaagaagaaaggggttcggaatcctgataggagtttacggaagctccgtaatttacatggtgcaactgccaatctttggagttatagacacgccttgctggatagtaaaagcggccccttcttgctcagaaaaaaagggaaactatgcttgccttttaagagaagatcaaggatggtattgtcagaatgcagggtcaactgtttactacccaaatgaaaaagactgcgaaacaagaggagaccatgtcttttgcgacacagcagcaggaatcaatgttgctgagcagtcaaaggagtgcaacatcaacatatccactactaattacccatgcaaagttagcacaggaagacaccctatcagtatggttgcactgtctcctcttggggctttggttgcttgctacaagggagtgagctgttccattggcagcaacagagtagggatcatcaagcaactgaacaaaggctgctcttatataaccaaccaagacgcagacacagtgacaatagacaacactgtataccagctaagcaaagttgagggcgaacagcatgttataaaaggaaggccagtgtcaagcagctttgatccagtcaaatttcctgaagatcaattcaatgttgcacttgaccaagttttcgaaagcattgagaacagtcaggccttggtggatcaatcaaacagaatcctaagcagtgcagagaaaggaaacactggcttcatcattgtaataattctaattgctgtccttggctctaccatgatcctagtgagtgtttttatcataataaagaaaacaaagaaacccacaggagcacctccagagctgagtggtgtcacaaacaatggcttcataccacataattagttaattaaaaataaagtaaattaaattaaaataaaataaaattaaaattaaaataaaataaaaataaaaatttgggacaaatcataatgtctcgcaaggctccatgcaaatatgaagtgcggggcaaatgcaatagaggaagtgagtgcaagtttaaccacaattactggagttggccagatagatacttactaataagatcaaattatttattaaatcaacttttaaggaacactgatagagctgatggcttatcaataatatcaggagcaggcagagaagataggacacaagattttgtcctaggttccaccaatgtggttcaaggttatattgatgataaccaaagcataacaaaagctgcagcctgttacagtctacataatataatcaaacaactacaagaagttgaagttaggcaggctagagataacaaaccatctgacagcaaacatgtggcacttcacaacttagtcctatcttatatggagatgagcaaaattcctgcatctttaatcaacaatctcaaaagactgccgagagagaaactgaaaaaattagcaaagcttataattgacttatcagcaggtgctgaaaatgactcttcatatgccttgcaagacagtgaaagcactaatcaagtgcagtgagcatggtcctgttttcattactatagaggttgattacatgatatggactcataaggacttaaaagaagctttatctaatgggatagtgaagtctcatactaacatttacaattgttatttagaaaacatagaaattatatatgtcaaggcttacttaagttagtaaaaacacatcagagtgggacaagtagttatggaggtgaaagtggagaacattcgaacaatagatatgctcaaagcaagagtgaaaaatcgtgtggcacgcagcaaatgctttaaaaatgcctctttgatcctaataggaataactacattgagtatagccctcaatatctatctaatcataaactatacaatgcaagaaaacacatccgaatcagaacatcacaccagctcatcacccatggaatccagcagggaaactccaacagtccctatggacaactcagacaccaatccaggctcacagtatccaactcaacagtccacagaaggctccacactctactttgcagcctcagcaagctcaccagagacagaaccaacatcaacaccagacacaacaagccgcccgcccttcgtcgacacacacacaacactaccaagtgcaagcagaacaaggacaagtccggcagtccacacaaaaaacaatccaaggacaagccccagaacacattccccaccatgggcaatgacaaggacggtccgtggaaccaccactctccgcacaagcagcacaagaaaaagaccgtccacagcatcagtccaacctgacagcagcgcaacaacccacaaacacgaagaagcaagcccagtgagcccgcaaacatctgcgagcacagcaagaccacaaaggaagggcatggaggccagcacatcaacaacatacaaccaaactagttaacaaaaaatacaaaataactctaagataaaccatatagacaccaacaattgagaagccaaaaggcaattcacaatctctccaaaaaggcaacaacaccatattagctccgcttaaatctccctggaaaaaacactcgcccatataccaactataccacaaccatcccaagaaaaaaagctgggtaaaacaacacccaagagacaaataacaatggatcctcttaatgaatccactgttaatgtctatcttcccgactcatatcttaaaggagtgatttcttttagtgagactaatgcaattggttcatgtctcttaaaaagaccttacctaaaaaatgacaacactgcaaaagttgccatagagaatcctgttatcgagcatgttagactcaaaaatgcaatcaattctaagatgaaaatatcagattacaggatagtagagccagtaaacatgcaacatgaaattatgaagaatgtacacagttgtgagctcacattattaaaacagtttttaacaaggagtaaaaatattagcactcttaaattaaatatgatatgtgattggctgcagttaaagtctacatcagatgatacctcaatcttaagttttatagatgtagaatttatacctagctgggtaagcaattggtttagtaattggtacaatctcaacaagttgattctggaattcaggaaagaagaagtaataagaactggttcaatcttgtgtaggtcattgggtaaattagtttttgttgtatcatcatacggatgtatagtcaagagcaacaaaagcaaaagagtgagcttcttcacatacaatcaactgttaacatggaaagatgtgatgttaagtagattcaatgcaaatttctgtatatgggtaagcaacagtctgaatgaaaatcaagaagggttagggttgagaagtaatctgcaaggcatattaactaataagctatatgaaactgtagattatatgcttagtttgtgttgcaatgaaggtttctcacttgtgaaagagtttgagggttttattatgagtgaaatccttaggattactgaacatgctcaattcagtactagatttagaaatactttattaaatggattaactgatcaattgacaaaattaaaaaataaaaacagactcagagttcatggtaccgtgttagaaaataatgattatccaatgtatgaagttgtacttaaattattaggagatactttgagatgtattaaattattaatcaataaaaacttagagaatgctgctgaattatactatatatttagaatattcggtcacccaatggtagatgaaagagatgcaatggatgctgtcaaattaaacaatgaaatcacaaaaatcctcaggttggagagcttgacagaactaagaggggcattcatattaaggattatcaaaggatttgtagacaacaacaaaagatggccgaaaattaaaaacttaaaagtgcttagtaaaagatggactatgtacttcaaagcaaaaagttaccctagtcaacttgaattaagtgaacaagattttttagagcttgctgcaatacagtttgaacaagagttttctgttcctgaaaaaaccaaccttgagatggtattaaatgataaagctatatcacctcctaaaagattaatttggtctgtgtacccaaaaaattacttacctgagacaataaaaaatcgatatctagaagagactttcaatgcaagtgatagtctcaaaacaagaagagtactagagtactatttgaaagataataaattcgaccaaaaagaacttaaaagttatgtggttaaacaagaatatttaaatgataaggatcatattgtctcgctaactggaaaagaaagagaattaagtgtaggtagaatgtttgctatgcaaccaggaaaacagcgacaaatacaaatattggctgaaaaattgttagctgataatattgtaccctttttcccagaaactttaacaaagtatggtgatctagatcttcagagaataatggaaatcaaatcagaactttcttctattaaaaccagaagaaatgatagttataataattacattgcaagagcatccatagtaacagatttaagtaagttcaaccaagcctttaggtatgaaactacagcgatctgtgcggatgtagcagatgaactacatggaacacaaagcctattctgttggttacatcttatcgttcctatgactacaatgatatgtgcctatagacatgcaccaccagaaacaaaaggtgaatatgatatagataagatagaagagcaaagtggtttatatagatatcatatgggtggtattgaaggatggtgtcaaaaactctggacaatggaagctatatctttattagatgttgtatctgtaaagacacgatgtcaaatgacatctttattaaacggtgacaaccaatcaatagatgtaagtaaaccagttaagttatctgagggtttagatgaagtgaaagcagattatagcttggctgtaaaaatgctaaaagaaataagagatgcatacagaaatataggccataaacttaaagaaggggaaacatatatatcaagagatcttcagtttataagtaaggtgattcaatctgaaggagtaatgcatcctacccctataaaaaagatcttaagagtgggaccatggataaacacaatattagatgacattaaaaccagtgcagagtcaatagggagtctatgtcaggaattagaatttaggggggaaagcataatagttagtctgatattaaggaatttttggctgtataatttatacatgcatgaatcaaagcaacaccccctagcagggaagcagttattcaaacaactaaataaaacattaacatcagtgcagagattttttgaaattaaaaaggaaaatgaagtagtagatctatggatgaacataccaatgcagtttggaggaggagatccagtagtcttctatagatctttctatagaaggacccctgattttttaactgaagcaatcagtcatgtagatattctgttaaaaatatcagccaacataagaaatgaagcgaaaataagtttcttcaaagccttactgtcaatagaaaaaaatgaacgtgctacactgacaacactaatgagagaccctcaagctgtgggctcagagcgacaagcaaaagtaacaagtgatatcaatagaacagcagttaccagcatcttaagtctttctccaaatcaacttttcagcgatagtgctatacactacagtagaaatgaagaagaggtcggaatcattgctgacaacataacacctgtttatcctcatggactgagagttttgtatgaatcattaccttttcataaagctgaaaaagttgtaaatatgatatcaggaacaaaatccataaccaacttattacagagaacatctgctattaatggtgaagatattgacagagctgtatccatgatgctggagaacctaggattattatctagaatattgtcagtagttgttgatagtatagaaattccaaccaaatctaatggtaggctgatatgttgtcagatatctagaaccctaagggagacatcatggaataatatggaaatagttggagtaacatcccctagcatcactacatgcatggatgtcatatatgcaactagctctcatttgaaagggataatcattgaaaagttcagcactgacagaactacaagaggtcaaagaggtccaaagagcccttgggtaggatcgagcacgcaagagaaaaaattagttcctgtttataacagacaaattctttcaaaacaacaaagagaacagctagaagcaattggaaaaatgagatgggtatataaagggacaccaggtttaagacgattactcaataagatttgtcttggaagtttaggcattagttacaaatgtgtaaaacctttattacctaggtttatgagtgtaaatttcctacacaggttatctgtcagtagtagacctatggaattcccagcatcagttccagcttatagaacaacaaattaccattttgacactagtcctattaatcaagcactaagtgaaagatttgggaatgaagatattaatttggtcttccaaaatgcaatcagctgtggaattagcataatgagtgtagtagaacaattaactggtaggagtccaaaacagttagttttaataccccaattagaagaaatagacattatgccaccaccagtgtttcaagggaaattcaattataagctagtagataagataacttctgaccaacatatcttcagtccagacaaaatagatatgttaacactggggaaaatgctcatgccaactataaaaggtcagaaaacagatcagttcttgaacaagagagagaattatttccatgggaacaatcttattgagtctttgtcagcagcgttagcatgtcattggtgtgggatattaacagagcaatgtatagaaaataatattttcaagaaagactggggtgacgggttcatatcggatcatgcttttatggacttcaaaatattcctatgtgtctttaaaactaaacttttatgtagttggggatcccaagggaaaaacattaaagatgaagatatagtagatgaatcgatagataaactgttaaggattgataatactttttggagaatgttcagcaaggttatgtttgaatcaaaggttaagaaaaggataatgttatatgatgtaaaatttctatcattagtaggttatatagggtttaagaattggtttatagaacagttgagatcagctgagttgcatgaggtaccttggattgtcaatgccgaaggtgatctggttgagatcaagtcaattaaaatctatttgcaactgatagagcagagtttatttttaagaataactgttttgaactatacagatatggcacatgctctcacaagattaatcagaaagaagttgatgtgtgataatgcactattaacttcaattccatccccaatggttaacttaactcaagttattgatcctacagaacaattagcttatttccctaagataacatttgaaaggctaaaaaattatgacactagttcaaattatgctaaaggaaagctaacaaggaattacatgatactgttgccatggcaacatgttaatagatataactttgtctttagttctactggatgtaaagttagtctaaaaacatgcattggaaaacttatgaaagatctaaaccctaaagttctgtactttattggagaaggggcaggaaattggatggccagaacagcatgtgaatatcctgacatcaaatttgtatacagaagtttaaaagatgaccttgatcatcattatcctttggaataccagagagtgataggagaattaagcaggataatagatagtggtgaagggctttcaatggaaacaacagatgcaactcaaaaaactcattgggatttgatacacagagtaagcaaagatgctttattaataactttatgtgatgcagaatttaaggacagagatgatttttttaagatggtaattctatggaggaaacatgtattatcatgcagaatttgcactacctatgggacagacctctatttattcgcaaagtatcatgctaaagactgcaatataaaattacctttttttgtgagatcagttgccacctttattatgcaaggtagtaaactgtcaggctcggaatgctacatactcttaacactaggccaccacaacaatttaccttgtcatggagaaatacaaaattctaagatgaaaatagcagcgtgtaatgatttttatgctgcaaaaaaacttgacaataaatcaattgaagccaactgtaaatcacttttatcagggctaagaataccgataaataagaaggaattaaatagacagagaaggttattaacactacaaagcaaccattcttctgtagcaacagttggaggtagcaaggtcatagagtctaaatggttaacaaacaaggcaaacacaataattgattggttagaacatattttaaattctccaaaaggtgaattaaattatgatttttttgaagcattagaaaatacttaccctaatatgattaaactaatagataatctagggaatgcagagattaaaaaactgatcaaagtaactggatatatgcttgtaagtaaaaaatgaaaaatgataaaaatgataaaataggtgacaacttcatactattccaaagtaatcatttgattatgcaattatgtaatagttaattaaaaactaaaaatcaaaagttaaaaactaataactgtcattaagtttattaaaaataagaaattataattggatgtatacggtttttttgccgt

SEQ ID NO: 3 - DNA, 12473 bp, metapneumovirus
gcgaaaaaaacgcgtataaattagattacaaaaaaatatgggacaagtgaaaatgtctcttcaagggattcacctgagtgatctatcatacaagcatgctatattaaaagagtctcagtacacaataaaaagagatgtgggtacaacaactgcagtgacaccctcatcattgcaacaagaaataacgctgttgtgtggagaaattctgtatgctaaacatgctgattacaaatatgctgcagaaataggaatacaatatattagcacagctttaggatcagagagagtgcagcagattctgaggaactcaggcagtgaagtccaagtggtcttaaccagaacgtactctctggggaaagttaaaaacaataaaggagaagatttacagatgttagacatacacggggtagagaagagctgggtagaagagatagacaaagaagcaaggaaaacaatggcaaccttgcttaaggaatcatcaggtaatatcccacaaaatcagaggccctcagcaccagacacacccataatcttattatgtgtaggtgcattaatatttactaagctagcatcaaccatagaagtgggactagagaccacagtcagaagggctaaccgtgtactaagtgatgcactcaagagataccctagaatggacatcccaaaaattgccagatccttctatgacttatttgaacaaaaagtgtatcacagaagtttgttcattgagtatggcaaagcattaggctcatcatctacaggcagcaaagcagaaagtctatttgttaatatattcatgcaagcttatggagccggtcaaacaatgctaaggtggggggtcattgccaggtcatccaacaatataatgttaggacatgtatctgtccaagctgagttaaaacaggtcacagaagtctatgacttggtgcgagaaatgggccctgaatctggacttctacatttaaggcaaagcccaaaagctggactgttatcactagccaactgtcccaactttgcaagtgttgttctcggaaatgcctcaggcttaggcataatcggtatgtatcgtgggagagtaccaaacacagaattattttcagcagcagaaagttatgccaaaagtttgaaagagagcaataaaatcaatttctcttcattaggacttacagatgaagagaaagaggctgcagaacatttcttaaatgtgagtgacgacagtcaaaatgattatgagtaattaaaaaagtgggacaagtcaaaatgtctttccctgaaggaaaagatattcttttcatgggtaatgaagcagcaaaattagcagaagctttccagaaatcattaaggaaaccaagtcataaaagatctcaatctattataggagaaaaagtgaacactgtatcagaaacattggaattacctactatcagtagacctgcaaaaccaaccatactgtcagaaccaaagttagcatggactgataaaggtggggcaatcaaaactgaaataaagcaagcaatcaaagtcatggatcctattgaggaagaagagtctactgagaagaaggtgctgccctccagtgatgggaaaacccctgcagaaaagaaactgaaaccatcaactaacaccaaaaagaaagtttcgtttacaccaaatgaaccaggaaaatatacaaagttggaaaaagatgctctagatttgctctcagataatgaagaagaagatgcagaatcttcaatcttaacctttgaagaaagagatacttcatcgttaagcattgaggccagattggaatcaatagaggagaaattaagcatgatattagggctattaagaacactcaacattgctacagcaggacccacagcagcaagagatgggatcagagatgcaatgattggcgtaagagaggaattaatagcagacataataaaggaagccaaagggaaagcagcagaaatgatggaagaggaaatgagtcaacgatcaaaaataggaaacggtagtgtaaaactaacagagaaagcaaaagagcttaacaaaattgttgaagatgaaagcacaagtggagaatctgaagaagaagaagaaccaaaagacatacaagacaatagtcaagaagatgacatttaccagttaattatgtagtttaataaaaataaacaatgggacaagtaaaaatggagtcctacctagtagacacttatcaaggcattccttacacagcagctgttcaagttgatctaatagaaaaggacctgttacctgcaagcctaacaatatggttccctttgtttcaggccaacacaccaccagcagtgctgctcgatcagttgaaaaccctaacaataaccactctgtatgctgcatcacaaaatggtccaatactcaaagtgaatgcatcagcccaaggtgcagcaatgtctgtacttcccaaaaaatttgaagtcaatgcgactgtagcactcgatgaatatagcaaattggaatttgacaaactcacagtctgtgaagtaaaaacagtttacttaacaaccatgaaaccatacgggatggtatcaaaatttgtgagctcagccaaatcagttggcaaaaaaacacatgatctaatcgcactgtgtgattttatggatctagaaaagaacacacctgttacaataccagcattcatcaaatcagtttcaatcaaagagagtgagtcagctactgttgaagctgctataagcagtgaagcagaccaagctctaacacaggccaaaattgcaccttatgcgggattgattatgatcatgactatgaacaatcccaaaggcatattcaaaaagcttggagctgggactcaagttatagtagaactaggagcatatgtccaggctgaaagcataagtaaaatatgcaagacttggagccatcaagggacaagatatgtgttgaagtccagataacagccaagcaccttggccaagagctactaactctatctcatagattataaagtcaccattctagttatataaaaatcaagttagaacaagaattaaatcaatcaagaatgggacaaataaaaatgtcttggaaagtggtgatcattttttcattgttaataacacctcaacacggtcttaaagagagctatttagaagagtcatgtagcactataactgaaggatatctcagtgttctgaggacaggttggtataccaacgtttttacactggaggtaggtgatgtagagaaccttacatgtgctgatggacctagcttaataaaaacagaattagacctgaccaaaagtgcactaagagaactcagaacagtttctgctgatcaactggcaagagaggagcaaattgagaatcccagacaatctagatttgttctaggagcaatagcactcggtgttgcaacagcagctgcagttacagcaggtgttgcaattgccaaaaccatccggcttgaaagtgaagtaacagcaattaagaatgccctcaaaaagaccaatgaagcagtatctacattggggaatggagttcgagtgttggcaactgcagtgagggagctggaagattttgtgagcaagaatctaacacgtgcaatcaacaaaaacaagtgcgacattgctgacctgaaaatggccgttagcttcagtcaattcaacagaaggtttctaaatgttgtgcggcaattttcagacaatgctggaataacaccagcaatatccttggacttaatgacagatgctgaactagccagagctgtttccaacatgccaacatctgcaggacaaataaaactgatgttggagaaccgtgcaatggtaagaagaaaggggttcggaatcctgataggagtttacggaagctccgtaatttacatggtgcaactgccaatctttggagttatagacacgccttgctggatagtaaaagcggccccttcttgctcagaaaaaaagggaaactatgcttgccttttaagagaagatcaaggatggtattgtcagaatgcagggtcaactgtttactacccaaatgaaaaagactgcgaaacaagaggagaccatgtcttttgcgacacagcagcaggaatcaatgttgctgagcagtcaaaggagtgcaacatcaacatatccactactaattacccatgcaaagttagcacaggaagacaccctatcagtatggttgcactgtctcctcttggggctttggttgcttgctacaagggagtgagctgttccattggcagcaacagagtagggatcatcaagcaactgaacaaaggctgctcttatataaccaaccaagacgcagacacagtgacaatagacaacactgtataccagctaagcaaagttgagggcgaacagcatgttataaaaggaaggccagtgtcaagcagctttgatccagtcaaatttcctgaagatcaattcaatgttgcacttgaccaagttttcgaaagcattgagaacagtcaggccttggtggatcaatcaaacagaatcctaagcagtgcagagaaaggaaacactggcttcatcattgtaataattctaattgctgtccttggctctaccatgatcctagtgagtgtttttatcataataaagaaaacaaagaaacccacaggagcacctccagagctgagtggtgtcacaaacaatggcttcataccacataattagttaattaaaaataaagtaaattaaattaaaataaaataaaattaaaattaaaataaaataaaaataaaaatttgggacaaatcataatgtctcgcaaggctccatgcaaatatgaagtgcggggcaaatgcaatagaggaagtgagtgcaagtttaaccacaattactggagttggccagatagatacttactaataagatcaaattatttattaaatcaacttttaaggaacactgatagagctgatggcttatcaataatatcaggagcaggcagagaagataggacacaagattttgtcctaggttccaccaatgtggttcaaggttatattgatgataaccaaagcataacaaaagctgcagcctgttacagtctacataatataatcaaacaactacaagaagttgaagttaggcaggctagagataacaaaccatctgacagcaaacatgtggcacttcacaacttagtcctatcttatatggagatgagcaaaattcctgcatctttaatcaacaatctcaaaagactgccgagagagaaactgaaaaaattagcaaagcttataattgacttatcagcaggtgctgaaaatgactcttcatatgccttgcaagacagtgaaagcactaatcaagtgcagtgagcatggtcctgttttcattactatagaggttgattacatgatatggactcataaggacttaaaagaagctttatctaatgggatagtgaagtctcatactaacatttacaattgttatttagaaaacatagaaattatatatgtcaaggcttacttaagttagtaaaaacacatcagagtgggataaatgacaatgataacattagatgtcattaaaagtgatgggtcttcaaaaacatgtactcacctcaaaaaattaattaaagaccactctggtaaagtgcttattgtacttaagttaatattagctttactaacatttctcacagtgacaatcaccatcaattatataaaagtagaaaacaatctgcaaatatgtcagtcaaaaactgaatcagacaaaaaggactcatcatcaaataccacatcagtcacaaccaagactactctaaatcatgatataacacagtattttaaaagtttgattcaaaggtatacaaactctgcaataaacagagacacatgctggaaaataagcagaaatcaatgcacaaacataacaacatacaaatttttatgttttaaatctgaagaaacaaaaaccaacaattgtgataaactgacagatttatgcagaaacaaaccaaaaccagctgttgaagtgtatcacatagtagaatgccattgtatatacacagttaaatggaagtgctatcattacccaatagatgaaacccaatcctaaataacactagattaggatccatccaagtctgttagttcaacaatttagttatttaaaaatattttgaaaacaagtaagtttctatgatacttcataataataagtaataattaattgcttaatcatcatcacaacattattcgaaaccataactattcaatttaagaagtaaaaacaataatatgagacaaataacaatggatcctcttaatgaatccactgttaatgtctatcttcccgactcatatcttaaaggagtgatttcttttagtgagactaatgcaattggttcatgtctcttaaaaagaccttacctaaaaaatgacaacactgcaaaagttgccatagagaatcctgttatcgagcatgttagactcaaaaatgcaatcaattctaagatgaaaatatcagattacaggatagtagagccagtaaacatgcaacatgaaattatgaagaatgtacacagttgtgagctcacattattaaaacagtttttaacaaggagtaaaaatattagcactcttaaattaaatatgatatgtgattggctgcagttaaagtctacatcagatgatacctcaatcttaagttttatagatgtagaatttatacctagctgggtaagcaattggtttagtaattggtacaatctcaacaagttgattctggaattcaggaaagaagaagtaataagaactggttcaatcttgtgtaggtcattgggtaaattagtttttgttgtatcatcatacggatgtatagtcaagagcaacaaaagcaaaagagtgagcttcttcacatacaatcaactgttaacatggaaagatgtgatgttaagtagattcaatgcaaatttctgtatatgggtaagcaacagtctgaatgaaaatcaagaagggttagggttgagaagtaatctgcaaggcatattaactaataagctatatgaaactgtagattatatgcttagtttgtgttgcaatgaaggtttctcacttgtgaaagagtttgagggttttattatgagtgaaatccttaggattactgaacatgctcaattcagtactagatttagaaatactttattaaatggattaactgatcaattgacaaaattaaaaaataaaaacagactcagagttcatggtaccgtgttagaaaataatgattatccaatgtatgaagttgtacttaaattattaggagatactttgagatgtattaaattattaatcaataaaaacttagagaatgctgctgaattatactatatatttagaatattcggtcacccaatggtagatgaaagagatgcaatggatgctgtcaaattaaacaatgaaatcacaaaaatcctcaggttggagagcttgacagaactaagaggggcattcatattaaggattatcaaaggatttgtagacaacaacaaaagatggccgaaaattaaaaacttaaaagtgcttagtaaaagatggactatgtacttcaaagcaaaaagttaccctagtcaacttgaattaagtgaacaagattttttagagcttgctgcaatacagtttgaacaagagttttctgttcctgaaaaaaccaaccttgagatggtattaaatgataaagctatatcacctcctaaaagattaatttggtctgtgtacccaaaaaattacttacctgagacaataaaaaatcgatatctagaagagactttcaatgcaagtgatagtctcaaaacaagaagagtactagagtactatttgaaagataataaattcgaccaaaaagaacttaaaagttatgtggttaaacaagaatatttaaatgataaggatcatattgtctcgctaactggaaaagaaagagaattaagtgtaggtagaatgtttgctatgcaaccaggaaaacagcgacaaatacaaatattggctgaaaaattgttagctgataatattgtaccctttttcccagaaactttaacaaagtatggtgatctagatcttcagagaataatggaaatcaaatcagaactttcttctattaaaaccagaagaaatgatagttataataattacattgcaagagcatccatagtaacagatttaagtaagttcaaccaagcctttaggtatgaaactacagcgatctgtgcggatgtagcagatgaactacatggaacacaaagcctattctgttggttacatcttatcgttcctatgactacaatgatatgtgcctatagacatgcaccaccagaaacaaaaggtgaatatgatatagataagatagaagagcaaagtggtttatatagatatcatatgggtggtattgaaggatggtgtcaaaaactctggacaatggaagctatatctttattagatgttgtatctgtaaagacacgatgtcaaatgacatctttattaaacggtgacaaccaatcaatagatgtaagtaaaccagttaagttatctgagggtttagatgaagtgaaagcagattatagcttggctgtaaaaatgctaaaagaaataagagatgcatacagaaatataggccataaacttaaagaaggggaaacatatatatcaagagatcttcagtttataagtaaggtgattcaatctgaaggagtaatgcatcctacccctataaaaaagatcttaagagtgggaccatggataaacacaatattagatgacattaaaaccagtgcagagtcaatagggagtctatgtcaggaattagaatttaggggggaaagcataatagttagtctgatattaaggaatttttggctgtataatttatacatgcatgaatcaaagcaacaccccctagcagggaagcagttattcaaacaactaaataaaacattaacatcagtgcagagattttttgaaattaaaaaggaaaatgaagtagtagatctatggatgaacataccaatgcagtttggaggaggagatccagtagtcttctatagatctttctatagaaggacccctgattttttaactgaagcaatcagtcatgtagatattctgttaaaaatatcagccaacataagaaatgaagcgaaaataagtttcttcaaagccttactgtcaatagaaaaaaatgaacgtgctacactgacaacactaatgagagaccctcaagctgtgggctcagagcgacaagcaaaagtaacaagtgatatcaatagaacagcagttaccagcatcttaagtctttctccaaatcaacttttcagcgatagtgctatacactacagtagaaatgaagaagaggtcggaatcattgctgacaacataacacctgtttatcctcatggactgagagttttgtatgaatcattaccttttcataaagctgaaaaagttgtaaatatgatatcaggaacaaaatccataaccaacttattacagagaacatctgctattaatggtgaagatattgacagagctgtatccatgatgctggagaacctaggattattatctagaatattgtcagtagttgttgatagtatagaaattccaaccaaatctaatggtaggctgatatgttgtcagatatctagaaccctaagggagacatcatggaataatatggaaatagttggagtaacatcccctagcatcactacatgcatggatgtcatatatgcaactagctctcatttgaaagggataatcattgaaaagttcagcactgacagaactacaagaggtcaaagaggtccaaagagcccttgggtaggatcgagcacgcaagagaaaaaattagttcctgtttataacagacaaattctttcaaaacaacaaagagaacagctagaagcaattggaaaaatgagatgggtatataaagggacaccaggtttaagacgattactcaataagatttgtcttggaagtttaggcattagttacaaatgtgtaaaacctttattacctaggtttatgagtgtaaatttcctacacaggttatctgtcagtagtagacctatggaattcccagcatcagttccagcttatagaacaacaaattaccattttgacactagtcctattaatcaagcactaagtgaaagatttgggaatgaagatattaatttggtcttccaaaatgcaatcagctgtggaattagcataatgagtgtagtagaacaattaactggtaggagtccaaaacagttagttttaataccccaattagaagaaatagacattatgccaccaccagtgtttcaagggaaattcaattataagctagtagataagataacttctgaccaacatatcttcagtccagacaaaatagatatgttaacactggggaaaatgctcatgccaactataaaaggtcagaaaacagatcagttcttgaacaagagagagaattatttccatgggaacaatcttattgagtctttgtcagcagcgttagcatgtcattggtgtgggatattaacagagcaatgtatagaaaataatattttcaagaaagactggggtgacgggttcatatcggatcatgcttttatggacttcaaaatattcctatgtgtctttaaaactaaacttttatgtagttggggatcccaagggaaaaacattaaagatgaagatatagtagatgaatcgatagataaactgttaaggattgataatactttttggagaatgttcagcaaggttatgtttgaatcaaaggttaagaaaaggataatgttatatgatgtaaaatttctatcattagtaggttatatagggtttaagaattggtttatagaacagttgagatcagctgagttgcatgaggtaccttggattgtcaatgccgaaggtgatctggttgagatcaagtcaattaaaatctatttgcaactgatagagcagagtttatttttaagaataactgttttgaactatacagatatggcacatgctctcacaagattaatcagaaagaagttgatgtgtgataatgcactattaacttcaattccatccccaatggttaacttaactcaagttattgatcctacagaacaattagcttatttccctaagataacatttgaaaggctaaaaaattatgacactagttcaaattatgctaaaggaaagctaacaaggaattacatgatactgttgccatggcaacatgttaatagatataactttgtctttagttctactggatgtaaagttagtctaaaaacatgcattggaaaacttatgaaagatctaaaccctaaagttctgtactttattggagaaggggcaggaaattggatggccagaacagcatgtgaatatcctgacatcaaatttgtatacagaagtttaaaagatgaccttgatcatcattatcctttggaataccagagagtgataggagaattaagcaggataatagatagtggtgaagggctttcaatggaaacaacagatgcaactcaaaaaactcattgggatttgatacacagagtaagcaaagatgctttattaataactttatgtgatgcagaatttaaggacagagatgatttttttaagatggtaattctatggaggaaacatgtattatcatgcagaatttgcactacctatgggacagacctctatttattcgcaaagtatcatgctaaagactgcaatataaaattacctttttttgtgagatcagttgccacctttattatgcaaggtagtaaactgtcaggctcggaatgctacatactcttaacactaggccaccacaacaatttaccttgtcatggagaaatacaaaattctaagatgaaaatagcagcgtgtaatgatttttatgctgcaaaaaaacttgacaataaatcaattgaagccaactgtaaatcacttttatcagggctaagaataccgataaataagaaggaattaaatagacagagaaggttattaacactacaaagcaaccattcttctgtagcaacagttggaggtagcaaggtcatagagtctaaatggttaacaaacaaggcaaacacaataattgattggttagaacatattttaaattctccaaaaggtgaattaaattatgatttttttgaagcattagaaaatacttaccctaatatgattaaactaatagataatctagggaatgcagagattaaaaaactgatcaaagtaactggatatatgcttgtaagtaaaaaatgaaaaatgataaaaatgataaaataggtgacaacttcatactattccaaagtaatcatttgattatgcaattatgtaatagttaattaaaaactaaaaatcaaaagttaaaaactaataactgtcattaagtttattaaaaataagaaattataattggatgtatacggtttttttgccgt

SEQ ID NO: 4 - DNA, 14141 bp, Artificial Sequence
  GFP coding sequence is inserted between (40) and (784)
gcgaaaaaaacgcgtataaattagattacaaaaaaatatgggacaagtgaaaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagttaattaaaaagtgggacaagtgaaaatgtctcttcaagggattcacctgagtgatctatcatacaagcatgctatattaaaagagtctcagtacacaataaaaagagatgtgggtacaacaactgcagtgacaccctcatcattgcaacaagaaataacgctgttgtgtggagaaattctgtatgctaaacatgctgattacaaatatgctgcagaaataggaatacaatatattagcacagctttaggatcagagagagtgcagcagattctgaggaactcaggcagtgaagtccaagtggtcttaaccagaacgtactctctggggaaagttaaaaacaataaaggagaagatttacagatgttagacatacacggggtagagaagagctgggtagaagagatagacaaagaagcaaggaaaacaatggcaaccttgcttaaggaatcatcaggtaatatcccacaaaatcagaggccctcagcaccagacacacccataatcttattatgtgtaggtgcattaatatttactaagctagcatcaaccatagaagtgggactagagaccacagtcagaagggctaaccgtgtactaagtgatgcactcaagagataccctagaatggacatcccaaaaattgccagatccttctatgacttatttgaacaaaaagtgtatcacagaagtttgttcattgagtatggcaaagcattaggctcatcatctacaggcagcaaagcagaaagtctatttgttaatatattcatgcaagcttatggagccggtcaaacaatgctaaggtggggggtcattgccaggtcatccaacaatataatgttaggacatgtatctgtccaagctgagttaaaacaggtcacagaagtctatgacttggtgcgagaaatgggccctgaatctggacttctacatttaaggcaaagcccaaaagctggactgttatcactagccaactgtcccaactttgcaagtgttgttctcggaaatgcctcaggcttaggcataatcggtatgtatcgtgggagagtaccaaacacagaattattttcagcagcagaaagttatgccaaaagtttgaaagagagcaataaaatcaatttctcttcattaggacttacagatgaagagaaagaggctgcagaacatttcttaaatgtgagtgacgacagtcaaaatgattatgagtaattaaaaaagtgggacaagtcaaaatgtctttccctgaaggaaaagatattcttttcatgggtaatgaagcagcaaaattagcagaagctttccagaaatcattaaggaaaccaagtcataaaagatctcaatctattataggagaaaaagtgaacactgtatcagaaacattggaattacctactatcagtagacctgcaaaaccaaccatactgtcagaaccaaagttagcatggactgataaaggtggggcaatcaaaactgaaataaagcaagcaatcaaagtcatggatcctattgaggaagaagagtctactgagaagaaggtgctgccctccagtgatgggaaaacccctgcagaaaagaaactgaaaccatcaactaacaccaaaaagaaagtttcgtttacaccaaatgaaccaggaaaatatacaaagttggaaaaagatgctctagatttgctctcagataatgaagaagaagatgcagaatcttcaatcttaacctttgaagaaagagatacttcatcgttaagcattgaggccagattggaatcaatagaggagaaattaagcatgatattagggctattaagaacactcaacattgctacagcaggacccacagcagcaagagatgggatcagagatgcaatgattggcgtaagagaggaattaatagcagacataataaaggaagccaaagggaaagcagcagaaatgatggaagaggaaatgagtcaacgatcaaaaataggaaacggtagtgtaaaactaacagagaaagcaaaagagcttaacaaaattgttgaagatgaaagcacaagtggagaatctgaagaagaagaagaaccaaaagacatacaagacaatagtcaagaagatgacatttaccagttaattatgtagtttaataaaaataaacaatgggacaagtaaaaatggagtcctacctagtagacacttatcaaggcattccttacacagcagctgttcaagttgatctaatagaaaaggacctgttacctgcaagcctaacaatatggttccctttgtttcaggccaacacaccaccagcagtgctgctcgatcagttgaaaaccctaacaataaccactctgtatgctgcatcacaaaatggtccaatactcaaagtgaatgcatcagcccaaggtgcagcaatgtctgtacttcccaaaaaatttgaagtcaatgcgactgtagcactcgatgaatatagcaaattggaatttgacaaactcacagtctgtgaagtaaaaacagtttacttaacaaccatgaaaccatacgggatggtatcaaaatttgtgagctcagccaaatcagttggcaaaaaaacacatgatctaatcgcactgtgtgattttatggatctagaaaagaacacacctgttacaataccagcattcatcaaatcagtttcaatcaaagagagtgagtcagctactgttgaagctgctataagcagtgaagcagaccaagctctaacacaggccaaaattgcaccttatgcgggattgattatgatcatgactatgaacaatcccaaaggcatattcaaaaagcttggagctgggactcaagttatagtagaactaggagcatatgtccaggctgaaagcataagtaaaatatgcaagacttggagccatcaagggacaagatatgtgttgaagtccagataacagccaagcaccttggccaagagctactaactctatctcatagattataaagtcaccattctagttatataaaaatcaagttagaacaagaattaaatcaatcaagaatgggacaaataaaaatgtcttggaaagtggtgatcattttttcattgttaataacacctcaacacggtcttaaagagagctatttagaagagtcatgtagcactataactgaaggatatctcagtgttctgaggacaggttggtataccaacgtttttacactggaggtaggtgatgtagagaaccttacatgtgctgatggacctagcttaataaaaacagaattagacctgaccaaaagtgcactaagagaactcagaacagtttctgctgatcaactggcaagagaggagcaaattgagaatcccagacaatctagatttgttctaggagcaatagcactcggtgttgcaacagcagctgcagttacagcaggtgttgcaattgccaaaaccatccggcttgaaagtgaagtaacagcaattaagaatgccctcaaaaagaccaatgaagcagtatctacattggggaatggagttcgagtgttggcaactgcagtgagggagctggaagattttgtgagcaagaatctaacacgtgcaatcaacaaaaacaagtgcgacattgctgacctgaaaatggccgttagcttcagtcaattcaacagaaggtttctaaatgttgtgcggcaattttcagacaatgctggaataacaccagcaatatccttggacttaatgacagatgctgaactagccagagctgtttccaacatgccaacatctgcaggacaaataaaactgatgttggagaaccgtgcaatggtaagaagaaaggggttcggaatcctgataggagtttacggaagctccgtaatttacatggtgcaactgccaatctttggagttatagacacgccttgctggatagtaaaagcggccccttcttgctcagaaaaaaagggaaactatgcttgccttttaagagaagatcaaggatggtattgtcagaatgcagggtcaactgtttactacccaaatgaaaaagactgcgaaacaagaggagaccatgtcttttgcgacacagcagcaggaatcaatgttgctgagcagtcaaaggagtgcaacatcaacatatccactactaattacccatgcaaagttagcacaggaagacaccctatcagtatggttgcactgtctcctcttggggctttggttgcttgctacaagggagtgagctgttccattggcagcaacagagtagggatcatcaagcaactgaacaaaggctgctcttatataaccaaccaagacgcagacacagtgacaatagacaacactgtataccagctaagcaaagttgagggcgaacagcatgttataaaaggaaggccagtgtcaagcagctttgatccagtcaaatttcctgaagatcaattcaatgttgcacttgaccaagttttcgaaagcattgagaacagtcaggccttggtggatcaatcaaacagaatcctaagcagtgcagagaaaggaaacactggcttcatcattgtaataattctaattgctgtccttggctctaccatgatcctagtgagtgtttttatcataataaagaaaacaaagaaacccacaggagcacctccagagctgagtggtgtcacaaacaatggcttcataccacataattagttaattaaaaataaagtaaattaaattaaaataaaataaaattaaaattaaaataaaataaaaataaaaatttgggacaaatcataatgtctcgcaaggctccatgcaaatatgaagtgcggggcaaatgcaatagaggaagtgagtgcaagtttaaccacaattactggagttggccagatagatacttactaataagatcaaattatttattaaatcaacttttaaggaacactgatagagctgatggcttatcaataatatcaggagcaggcagagaagataggacacaagattttgtcctaggttccaccaatgtggttcaaggttatattgatgataaccaaagcataacaaaagctgcagcctgttacagtctacataatataatcaaacaactacaagaagttgaagttaggcaggctagagataacaaaccatctgacagcaaacatgtggcacttcacaacttagtcctatcttatatggagatgagcaaaattcctgcatctttaatcaacaatctcaaaagactgccgagagagaaactgaaaaaattagcaaagcttataattgacttatcagcaggtgctgaaaatgactcttcatatgccttgcaagacagtgaaagcactaatcaagtgcagtgagcatggtcctgttttcattactatagaggttgattacatgatatggactcataaggacttaaaagaagctttatctaatgggatagtgaagtctcatactaacatttacaattgttatttagaaaacatagaaattatatatgtcaaggcttacttaagttagtaaaaacacatcagagtgggataaatgacaatgataacattagatgtcattaaaagtgatgggtcttcaaaaacatgtactcacctcaaaaaattaattaaagaccactctggtaaagtgcttattgtacttaagttaatattagctttactaacatttctcacagtgacaatcaccatcaattatataaaagtagaaaacaatctgcaaatatgtcagtcaaaaactgaatcagacaaaaaggactcatcatcaaataccacatcagtcacaaccaagactactctaaatcatgatataacacagtattttaaaagtttgattcaaaggtatacaaactctgcaataaacagagacacatgctggaaaataagcagaaatcaatgcacaaacataacaacatacaaatttttatgttttaaatctgaagaaacaaaaaccaacaattgtgataaactgacagatttatgcagaaacaaaccaaaaccagctgttgaagtgtatcacatagtagaatgccattgtatatacacagttaaatggaagtgctatcattacccaatagatgaaacccaatcctaaataacactagattaggatccatccaagtctgttagttcaacaatttagttatttaaaaatattttgaaaacaagtaagtttctatgatacttcataataataagtaataattaattgcttaatcatcatcacaacattattcgaaaccataactattcaatttaagaagtaaaaacaataatatgggacaagtagttatggaggtgaaagtggagaacattcgaacaatagatatgctcaaagcaagagtgaaaaatcgtgtggcacgcagcaaatgctttaaaaatgcctctttgatcctaataggaataactacattgagtatagccctcaatatctatctaatcataaactatacaatgcaagaaaacacatccgaatcagaacatcacaccagctcatcacccatggaatccagcagggaaactccaacagtccctatggacaactcagacaccaatccaggctcacagtatccaactcaacagtccacagaaggctccacactctactttgcagcctcagcaagctcaccagagacagaaccaacatcaacaccagacacaacaagccgcccgcccttcgtcgacacacacacaacactaccaagtgcaagcagaacaaggacaagtccggcagtccacacaaaaaacaatccaaggacaagccccagaacacattccccaccatgggcaatgacaaggacggtccgtggaaccaccactctccgcacaagcagcacaagaaaaagaccgtccacagcatcagtccaacctgacagcagcgcaacaacccacaaacacgaagaagcaagcccagtgagcccgcaaacatctgcgagcacagcaagaccacaaaggaagggcatggaggccagcacatcaacaacatacaaccaaactagttaacaaaaaatacaaaataactctaagataaaccatatagacaccaacaattgagaagccaaaaggcaattcacaatctctccaaaaaggcaacaacaccatattagctccgcttaaatctccctggaaaaaacactcgcccatataccaactataccacaaccatcccaagaaaaaaagctgggtaaaacaacacccaagagacaaataacaatggatcctcttaatgaatccactgttaatgtctatcttcccgactcatatcttaaaggagtgatttcttttagtgagactaatgcaattggttcatgtctcttaaaaagaccttacctaaaaaatgacaacactgcaaaagttgccatagagaatcctgttatcgagcatgttagactcaaaaatgcaatcaattctaagatgaaaatatcagattacaggatagtagagccagtaaacatgcaacatgaaattatgaagaatgtacacagttgtgagctcacattattaaaacagtttttaacaaggagtaaaaatattagcactcttaaattaaatatgatatgtgattggctgcagttaaagtctacatcagatgatacctcaatcttaagttttatagatgtagaatttatacctagctgggtaagcaattggtttagtaattggtacaatctcaacaagttgattctggaattcaggaaagaagaagtaataagaactggttcaatcttgtgtaggtcattgggtaaattagtttttgttgtatcatcatacggatgtatagtcaagagcaacaaaagcaaaagagtgagcttcttcacatacaatcaactgttaacatggaaagatgtgatgttaagtagattcaatgcaaatttctgtatatgggtaagcaacagtctgaatgaaaatcaagaagggttagggttgagaagtaatctgcaaggcatattaactaataagctatatgaaactgtagattatatgcttagtttgtgttgcaatgaaggtttctcacttgtgaaagagtttgagggttttattatgagtgaaatccttaggattactgaacatgctcaattcagtactagatttagaaatactttattaaatggattaactgatcaattgacaaaattaaaaaataaaaacagactcagagttcatggtaccgtgttagaaaataatgattatccaatgtatgaagttgtacttaaattattaggagatactttgagatgtattaaattattaatcaataaaaacttagagaatgctgctgaattatactatatatttagaatattcggtcacccaatggtagatgaaagagatgcaatggatgctgtcaaattaaacaatgaaatcacaaaaatcctcaggttggagagcttgacagaactaagaggggcattcatattaaggattatcaaaggatttgtagacaacaacaaaagatggccgaaaattaaaaacttaaaagtgcttagtaaaagatggactatgtacttcaaagcaaaaagttaccctagtcaacttgaattaagtgaacaagattttttagagcttgctgcaatacagtttgaacaagagttttctgttcctgaaaaaaccaaccttgagatggtattaaatgataaagctatatcacctcctaaaagattaatttggtctgtgtacccaaaaaattacttacctgagacaataaaaaatcgatatctagaagagactttcaatgcaagtgatagtctcaaaacaagaagagtactagagtactatttgaaagataataaattcgaccaaaaagaacttaaaagttatgtggttaaacaagaatatttaaatgataaggatcatattgtctcgctaactggaaaagaaagagaattaagtgtaggtagaatgtttgctatgcaaccaggaaaacagcgacaaatacaaatattggctgaaaaattgttagctgataatattgtaccctttttcccagaaactttaacaaagtatggtgatctagatcttcagagaataatggaaatcaaatcagaactttcttctattaaaaccagaagaaatgatagttataataattacattgcaagagcatccatagtaacagatttaagtaagttcaaccaagcctttaggtatgaaactacagcgatctgtgcggatgtagcagatgaactacatggaacacaaagcctattctgttggttacatcttatcgttcctatgactacaatgatatgtgcctatagacatgcaccaccagaaacaaaaggtgaatatgatatagataagatagaagagcaaagtggtttatatagatatcatatgggtggtattgaaggatggtgtcaaaaactctggacaatggaagctatatctttattagatgttgtatctgtaaagacacgatgtcaaatgacatctttattaaacggtgacaaccaatcaatagatgtaagtaaaccagttaagttatctgagggtttagatgaagtgaaagcagattatagcttggctgtaaaaatgctaaaagaaataagagatgcatacagaaatataggccataaacttaaagaaggggaaacatatatatcaagagatcttcagtttataagtaaggtgattcaatctgaaggagtaatgcatcctacccctataaaaaagatcttaagagtgggaccatggataaacacaatattagatgacattaaaaccagtgcagagtcaatagggagtctatgtcaggaattagaatttaggggggaaagcataatagttagtctgatattaaggaatttttggctgtataatttatacatgcatgaatcaaagcaacaccccctagcagggaagcagttattcaaacaactaaataaaacattaacatcagtgcagagattttttgaaattaaaaaggaaaatgaagtagtagatctatggatgaacataccaatgcagtttggaggaggagatccagtagtcttctatagatctttctatagaaggacccctgattttttaactgaagcaatcagtcatgtagatattctgttaaaaatatcagccaacataagaaatgaagcgaaaataagtttcttcaaagccttactgtcaatagaaaaaaatgaacgtgctacactgacaacactaatgagagaccctcaagctgtgggctcagagcgacaagcaaaagtaacaagtgatatcaatagaacagcagttaccagcatcttaagtctttctccaaatcaacttttcagcgatagtgctatacactacagtagaaatgaagaagaggtcggaatcattgctgacaacataacacctgtttatcctcatggactgagagttttgtatgaatcattaccttttcataaagctgaaaaagttgtaaatatgatatcaggaacaaaatccataaccaacttattacagagaacatctgctattaatggtgaagatattgacagagctgtatccatgatgctggagaacctaggattattatctagaatattgtcagtagttgttgatagtatagaaattccaaccaaatctaatggtaggctgatatgttgtcagatatctagaaccctaagggagacatcatggaataatatggaaatagttggagtaacatcccctagcatcactacatgcatggatgtcatatatgcaactagctctcatttgaaagggataatcattgaaaagttcagcactgacagaactacaagaggtcaaagaggtccaaagagcccttgggtaggatcgagcacgcaagagaaaaaattagttcctgtttataacagacaaattctttcaaaacaacaaagagaacagctagaagcaattggaaaaatgagatgggtatataaagggacaccaggtttaagacgattactcaataagatttgtcttggaagtttaggcattagttacaaatgtgtaaaacctttattacctaggtttatgagtgtaaatttcctacacaggttatctgtcagtagtagacctatggaattcccagcatcagttccagcttatagaacaacaaattaccattttgacactagtcctattaatcaagcactaagtgaaagatttgggaatgaagatattaatttggtcttccaaaatgcaatcagctgtggaattagcataatgagtgtagtagaacaattaactggtaggagtccaaaacagttagttttaataccccaattagaagaaatagacattatgccaccaccagtgtttcaagggaaattcaattataagctagtagataagataacttctgaccaacatatcttcagtccagacaaaatagatatgttaacactggggaaaatgctcatgccaactataaaaggtcagaaaacagatcagttcttgaacaagagagagaattatttccatgggaacaatcttattgagtctttgtcagcagcgttagcatgtcattggtgtgggatattaacagagcaatgtatagaaaataatattttcaagaaagactggggtgacgggttcatatcggatcatgcttttatggacttcaaaatattcctatgtgtctttaaaactaaacttttatgtagttggggatcccaagggaaaaacattaaagatgaagatatagtagatgaatcgatagataaactgttaaggattgataatactttttggagaatgttcagcaaggttatgtttgaatcaaaggttaagaaaaggataatgttatatgatgtaaaatttctatcattagtaggttatatagggtttaagaattggtttatagaacagttgagatcagctgagttgcatgaggtaccttggattgtcaatgccgaaggtgatctggttgagatcaagtcaattaaaatctatttgcaactgatagagcagagtttatttttaagaataactgttttgaactatacagatatggcacatgctctcacaagattaatcagaaagaagttgatgtgtgataatgcactattaacttcaattccatccccaatggttaacttaactcaagttattgatcctacagaacaattagcttatttccctaagataacatttgaaaggctaaaaaattatgacactagttcaaattatgctaaaggaaagctaacaaggaattacatgatactgttgccatggcaacatgttaatagatataactttgtctttagttctactggatgtaaagttagtctaaaaacatgcattggaaaacttatgaaagatctaaaccctaaagttctgtactttattggagaaggggcaggaaattggatggccagaacagcatgtgaatatcctgacatcaaatttgtatacagaagtttaaaagatgaccttgatcatcattatcctttggaataccagagagtgataggagaattaagcaggataatagatagtggtgaagggctttcaatggaaacaacagatgcaactcaaaaaactcattgggatttgatacacagagtaagcaaagatgctttattaataactttatgtgatgcagaatttaaggacagagatgatttttttaagatggtaattctatggaggaaacatgtattatcatgcagaatttgcactacctatgggacagacctctatttattcgcaaagtatcatgctaaagactgcaatataaaattacctttttttgtgagatcagttgccacctttattatgcaaggtagtaaactgtcaggctcggaatgctacatactcttaacactaggccaccacaacaatttaccttgtcatggagaaatacaaaattctaagatgaaaatagcagcgtgtaatgatttttatgctgcaaaaaaacttgacaataaatcaattgaagccaactgtaaatcacttttatcagggctaagaataccgataaataagaaggaattaaatagacagagaaggttattaacactacaaagcaaccattcttctgtagcaacagttggaggtagcaaggtcatagagtctaaatggttaacaaacaaggcaaacacaataattgattggttagaacatattttaaattctccaaaaggtgaattaaattatgatttttttgaagcattagaaaatacttaccctaatatgattaaactaatagataatctagggaatgcagagattaaaaaactgatcaaagtaactggatatatgcttgtaagtaaaaaatgaaaaatgataaaaatgataaaataggtgacaacttcatactattccaaagtaatcatttgattatgcaattatgtaatagttaattaaaaactaaaaatcaaaagttaaaaactaataactgtcattaagtttattaaaaataagaaattataattggatgtatacggtttttttgccgt

SEQ ID NO: 5 - DNA, 13392 bp, Artificial Sequence
  GFP coding sequence is inserted between (40) and (784)
gcgaaaaaaacgcgtataaattagattacaaaaaaatatgggacaagtgaaaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagttaattaaaaagtgggacaagtgaaaatgtctcttcaagggattcacctgagtgatctatcatacaagcatgctatattaaaagagtctcagtacacaataaaaagagatgtgggtacaacaactgcagtgacaccctcatcattgcaacaagaaataacgctgttgtgtggagaaattctgtatgctaaacatgctgattacaaatatgctgcagaaataggaatacaatatattagcacagctttaggatcagagagagtgcagcagattctgaggaactcaggcagtgaagtccaagtggtcttaaccagaacgtactctctggggaaagttaaaaacaataaaggagaagatttacagatgttagacatacacggggtagagaagagctgggtagaagagatagacaaagaagcaaggaaaacaatggcaaccttgcttaaggaatcatcaggtaatatcccacaaaatcagaggccctcagcaccagacacacccataatcttattatgtgtaggtgcattaatatttactaagctagcatcaaccatagaagtgggactagagaccacagtcagaagggctaaccgtgtactaagtgatgcactcaagagataccctagaatggacatcccaaaaattgccagatccttctatgacttatttgaacaaaaagtgtatcacagaagtttgttcattgagtatggcaaagcattaggctcatcatctacaggcagcaaagcagaaagtctatttgttaatatattcatgcaagcttatggagccggtcaaacaatgctaaggtggggggtcattgccaggtcatccaacaatataatgttaggacatgtatctgtccaagctgagttaaaacaggtcacagaagtctatgacttggtgcgagaaatgggccctgaatctggacttctacatttaaggcaaagcccaaaagctggactgttatcactagccaactgtcccaactttgcaagtgttgttctcggaaatgcctcaggcttaggcataatcggtatgtatcgtgggagagtaccaaacacagaattattttcagcagcagaaagttatgccaaaagtttgaaagagagcaataaaatcaatttctcttcattaggacttacagatgaagagaaagaggctgcagaacatttcttaaatgtgagtgacgacagtcaaaatgattatgagtaattaaaaaagtgggacaagtcaaaatgtctttccctgaaggaaaagatattcttttcatgggtaatgaagcagcaaaattagcagaagctttccagaaatcattaaggaaaccaagtcataaaagatctcaatctattataggagaaaaagtgaacactgtatcagaaacattggaattacctactatcagtagacctgcaaaaccaaccatactgtcagaaccaaagttagcatggactgataaaggtggggcaatcaaaactgaaataaagcaagcaatcaaagtcatggatcctattgaggaagaagagtctactgagaagaaggtgctgccctccagtgatgggaaaacccctgcagaaaagaaactgaaaccatcaactaacaccaaaaagaaagtttcgtttacaccaaatgaaccaggaaaatatacaaagttggaaaaagatgctctagatttgctctcagataatgaagaagaagatgcagaatcttcaatcttaacctttgaagaaagagatacttcatcgttaagcattgaggccagattggaatcaatagaggagaaattaagcatgatattagggctattaagaacactcaacattgctacagcaggacccacagcagcaagagatgggatcagagatgcaatgattggcgtaagagaggaattaatagcagacataataaaggaagccaaagggaaagcagcagaaatgatggaagaggaaatgagtcaacgatcaaaaataggaaacggtagtgtaaaactaacagagaaagcaaaagagcttaacaaaattgttgaagatgaaagcacaagtggagaatctgaagaagaagaagaaccaaaagacatacaagacaatagtcaagaagatgacatttaccagttaattatgtagtttaataaaaataaacaatgggacaagtaaaaatggagtcctacctagtagacacttatcaaggcattccttacacagcagctgttcaagttgatctaatagaaaaggacctgttacctgcaagcctaacaatatggttccctttgtttcaggccaacacaccaccagcagtgctgctcgatcagttgaaaaccctaacaataaccactctgtatgctgcatcacaaaatggtccaatactcaaagtgaatgcatcagcccaaggtgcagcaatgtctgtacttcccaaaaaatttgaagtcaatgcgactgtagcactcgatgaatatagcaaattggaatttgacaaactcacagtctgtgaagtaaaaacagtttacttaacaaccatgaaaccatacgggatggtatcaaaatttgtgagctcagccaaatcagttggcaaaaaaacacatgatctaatcgcactgtgtgattttatggatctagaaaagaacacacctgttacaataccagcattcatcaaatcagtttcaatcaaagagagtgagtcagctactgttgaagctgctataagcagtgaagcagaccaagctctaacacaggccaaaattgcaccttatgcgggattgattatgatcatgactatgaacaatcccaaaggcatattcaaaaagcttggagctgggactcaagttatagtagaactaggagcatatgtccaggctgaaagcataagtaaaatatgcaagacttggagccatcaagggacaagatatgtgttgaagtccagataacagccaagcaccttggccaagagctactaactctatctcatagattataaagtcaccattctagttatataaaaatcaagttagaacaagaattaaatcaatcaagaatgggacaaataaaaatgtcttggaaagtggtgatcattttttcattgttaataacacctcaacacggtcttaaagagagctatttagaagagtcatgtagcactataactgaaggatatctcagtgttctgaggacaggttggtataccaacgtttttacactggaggtaggtgatgtagagaaccttacatgtgctgatggacctagcttaataaaaacagaattagacctgaccaaaagtgcactaagagaactcagaacagtttctgctgatcaactggcaagagaggagcaaattgagaatcccagacaatctagatttgttctaggagcaatagcactcggtgttgcaacagcagctgcagttacagcaggtgttgcaattgccaaaaccatccggcttgaaagtgaagtaacagcaattaagaatgccctcaaaaagaccaatgaagcagtatctacattggggaatggagttcgagtgttggcaactgcagtgagggagctggaagattttgtgagcaagaatctaacacgtgcaatcaacaaaaacaagtgcgacattgctgacctgaaaatggccgttagcttcagtcaattcaacagaaggtttctaaatgttgtgcggcaattttcagacaatgctggaataacaccagcaatatccttggacttaatgacagatgctgaactagccagagctgtttccaacatgccaacatctgcaggacaaataaaactgatgttggagaaccgtgcaatggtaagaagaaaggggttcggaatcctgataggagtttacggaagctccgtaatttacatggtgcaactgccaatctttggagttatagacacgccttgctggatagtaaaagcggccccttcttgctcagaaaaaaagggaaactatgcttgccttttaagagaagatcaaggatggtattgtcagaatgcagggtcaactgtttactacccaaatgaaaaagactgcgaaacaagaggagaccatgtcttttgcgacacagcagcaggaatcaatgttgctgagcagtcaaaggagtgcaacatcaacatatccactactaattacccatgcaaagttagcacaggaagacaccctatcagtatggttgcactgtctcctcttggggctttggttgcttgctacaagggagtgagctgttccattggcagcaacagagtagggatcatcaagcaactgaacaaaggctgctcttatataaccaaccaagacgcagacacagtgacaatagacaacactgtataccagctaagcaaagttgagggcgaacagcatgttataaaaggaaggccagtgtcaagcagctttgatccagtcaaatttcctgaagatcaattcaatgttgcacttgaccaagttttcgaaagcattgagaacagtcaggccttggtggatcaatcaaacagaatcctaagcagtgcagagaaaggaaacactggcttcatcattgtaataattctaattgctgtccttggctctaccatgatcctagtgagtgtttttatcataataaagaaaacaaagaaacccacaggagcacctccagagctgagtggtgtcacaaacaatggcttcataccacataattagttaattaaaaataaagtaaattaaattaaaataaaataaaattaaaattaaaataaaataaaaataaaaatttgggacaaatcataatgtctcgcaaggctccatgcaaatatgaagtgcggggcaaatgcaatagaggaagtgagtgcaagtttaaccacaattactggagttggccagatagatacttactaataagatcaaattatttattaaatcaacttttaaggaacactgatagagctgatggcttatcaataatatcaggagcaggcagagaagataggacacaagattttgtcctaggttccaccaatgtggttcaaggttatattgatgataaccaaagcataacaaaagctgcagcctgttacagtctacataatataatcaaacaactacaagaagttgaagttaggcaggctagagataacaaaccatctgacagcaaacatgtggcacttcacaacttagtcctatcttatatggagatgagcaaaattcctgcatctttaatcaacaatctcaaaagactgccgagagagaaactgaaaaaattagcaaagcttataattgacttatcagcaggtgctgaaaatgactcttcatatgccttgcaagacagtgaaagcactaatcaagtgcagtgagcatggtcctgttttcattactatagaggttgattacatgatatggactcataaggacttaaaagaagctttatctaatgggatagtgaagtctcatactaacatttacaattgttatttagaaaacatagaaattatatatgtcaaggcttacttaagttagtaaaaacacatcagagtgggacaagtagttatggaggtgaaagtggagaacattcgaacaatagatatgctcaaagcaagagtgaaaaatcgtgtggcacgcagcaaatgctttaaaaatgcctctttgatcctaataggaataactacattgagtatagccctcaatatctatctaatcataaactatacaatgcaagaaaacacatccgaatcagaacatcacaccagctcatcacccatggaatccagcagggaaactccaacagtccctatggacaactcagacaccaatccaggctcacagtatccaactcaacagtccacagaaggctccacactctactttgcagcctcagcaagctcaccagagacagaaccaacatcaacaccagacacaacaagccgcccgcccttcgtcgacacacacacaacactaccaagtgcaagcagaacaaggacaagtccggcagtccacacaaaaaacaatccaaggacaagccccagaacacattccccaccatgggcaatgacaaggacggtccgtggaaccaccactctccgcacaagcagcacaagaaaaagaccgtccacagcatcagtccaacctgacagcagcgcaacaacccacaaacacgaagaagcaagcccagtgagcccgcaaacatctgcgagcacagcaagaccacaaaggaagggcatggaggccagcacatcaacaacatacaaccaaactagttaacaaaaaatacaaaataactctaagataaaccatatagacaccaacaattgagaagccaaaaggcaattcacaatctctccaaaaaggcaacaacaccatattagctccgcttaaatctccctggaaaaaacactcgcccatataccaactataccacaaccatcccaagaaaaaaagctgggtaaaacaacacccaagagacaaataacaatggatcctcttaatgaatccactgttaatgtctatcttcccgactcatatcttaaaggagtgatttcttttagtgagactaatgcaattggttcatgtctcttaaaaagaccttacctaaaaaatgacaacactgcaaaagttgccatagagaatcctgttatcgagcatgttagactcaaaaatgcaatcaattctaagatgaaaatatcagattacaggatagtagagccagtaaacatgcaacatgaaattatgaagaatgtacacagttgtgagctcacattattaaaacagtttttaacaaggagtaaaaatattagcactcttaaattaaatatgatatgtgattggctgcagttaaagtctacatcagatgatacctcaatcttaagttttatagatgtagaatttatacctagctgggtaagcaattggtttagtaattggtacaatctcaacaagttgattctggaattcaggaaagaagaagtaataagaactggttcaatcttgtgtaggtcattgggtaaattagtttttgttgtatcatcatacggatgtatagtcaagagcaacaaaagcaaaagagtgagcttcttcacatacaatcaactgttaacatggaaagatgtgatgttaagtagattcaatgcaaatttctgtatatgggtaagcaacagtctgaatgaaaatcaagaagggttagggttgagaagtaatctgcaaggcatattaactaataagctatatgaaactgtagattatatgcttagtttgtgttgcaatgaaggtttctcacttgtgaaagagtttgagggttttattatgagtgaaatccttaggattactgaacatgctcaattcagtactagatttagaaatactttattaaatggattaactgatcaattgacaaaattaaaaaataaaaacagactcagagttcatggtaccgtgttagaaaataatgattatccaatgtatgaagttgtacttaaattattaggagatactttgagatgtattaaattattaatcaataaaaacttagagaatgctgctgaattatactatatatttagaatattcggtcacccaatggtagatgaaagagatgcaatggatgctgtcaaattaaacaatgaaatcacaaaaatcctcaggttggagagcttgacagaactaagaggggcattcatattaaggattatcaaaggatttgtagacaacaacaaaagatggccgaaaattaaaaacttaaaagtgcttagtaaaagatggactatgtacttcaaagcaaaaagttaccctagtcaacttgaattaagtgaacaagattttttagagcttgctgcaatacagtttgaacaagagttttctgttcctgaaaaaaccaaccttgagatggtattaaatgataaagctatatcacctcctaaaagattaatttggtctgtgtacccaaaaaattacttacctgagacaataaaaaatcgatatctagaagagactttcaatgcaagtgatagtctcaaaacaagaagagtactagagtactatttgaaagataataaattcgaccaaaaagaacttaaaagttatgtggttaaacaagaatatttaaatgataaggatcatattgtctcgctaactggaaaagaaagagaattaagtgtaggtagaatgtttgctatgcaaccaggaaaacagcgacaaatacaaatattggctgaaaaattgttagctgataatattgtaccctttttcccagaaactttaacaaagtatggtgatctagatcttcagagaataatggaaatcaaatcagaactttcttctattaaaaccagaagaaatgatagttataataattacattgcaagagcatccatagtaacagatttaagtaagttcaaccaagcctttaggtatgaaactacagcgatctgtgcggatgtagcagatgaactacatggaacacaaagcctattctgttggttacatcttatcgttcctatgactacaatgatatgtgcctatagacatgcaccaccagaaacaaaaggtgaatatgatatagataagatagaagagcaaagtggtttatatagatatcatatgggtggtattgaaggatggtgtcaaaaactctggacaatggaagctatatctttattagatgttgtatctgtaaagacacgatgtcaaatgacatctttattaaacggtgacaaccaatcaatagatgtaagtaaaccagttaagttatctgagggtttagatgaagtgaaagcagattatagcttggctgtaaaaatgctaaaagaaataagagatgcatacagaaatataggccataaacttaaagaaggggaaacatatatatcaagagatcttcagtttataagtaaggtgattcaatctgaaggagtaatgcatcctacccctataaaaaagatcttaagagtgggaccatggataaacacaatattagatgacattaaaaccagtgcagagtcaatagggagtctatgtcaggaattagaatttaggggggaaagcataatagttagtctgatattaaggaatttttggctgtataatttatacatgcatgaatcaaagcaacaccccctagcagggaagcagttattcaaacaactaaataaaacattaacatcagtgcagagattttttgaaattaaaaaggaaaatgaagtagtagatctatggatgaacataccaatgcagtttggaggaggagatccagtagtcttctatagatctttctatagaaggacccctgattttttaactgaagcaatcagtcatgtagatattctgttaaaaatatcagccaacataagaaatgaagcgaaaataagtttcttcaaagccttactgtcaatagaaaaaaatgaacgtgctacactgacaacactaatgagagaccctcaagctgtgggctcagagcgacaagcaaaagtaacaagtgatatcaatagaacagcagttaccagcatcttaagtctttctccaaatcaacttttcagcgatagtgctatacactacagtagaaatgaagaagaggtcggaatcattgctgacaacataacacctgtttatcctcatggactgagagttttgtatgaatcattaccttttcataaagctgaaaaagttgtaaatatgatatcaggaacaaaatccataaccaacttattacagagaacatctgctattaatggtgaagatattgacagagctgtatccatgatgctggagaacctaggattattatctagaatattgtcagtagttgttgatagtatagaaattccaaccaaatctaatggtaggctgatatgttgtcagatatctagaaccctaagggagacatcatggaataatatggaaatagttggagtaacatcccctagcatcactacatgcatggatgtcatatatgcaactagctctcatttgaaagggataatcattgaaaagttcagcactgacagaactacaagaggtcaaagaggtccaaagagcccttgggtaggatcgagcacgcaagagaaaaaattagttcctgtttataacagacaaattctttcaaaacaacaaagagaacagctagaagcaattggaaaaatgagatgggtatataaagggacaccaggtttaagacgattactcaataagatttgtcttggaagtttaggcattagttacaaatgtgtaaaacctttattacctaggtttatgagtgtaaatttcctacacaggttatctgtcagtagtagacctatggaattcccagcatcagttccagcttatagaacaacaaattaccattttgacactagtcctattaatcaagcactaagtgaaagatttgggaatgaagatattaatttggtcttccaaaatgcaatcagctgtggaattagcataatgagtgtagtagaacaattaactggtaggagtccaaaacagttagttttaataccccaattagaagaaatagacattatgccaccaccagtgtttcaagggaaattcaattataagctagtagataagataacttctgaccaacatatcttcagtccagacaaaatagatatgttaacactggggaaaatgctcatgccaactataaaaggtcagaaaacagatcagttcttgaacaagagagagaattatttccatgggaacaatcttattgagtctttgtcagcagcgttagcatgtcattggtgtgggatattaacagagcaatgtatagaaaataatattttcaagaaagactggggtgacgggttcatatcggatcatgcttttatggacttcaaaatattcctatgtgtctttaaaactaaacttttatgtagttggggatcccaagggaaaaacattaaagatgaagatatagtagatgaatcgatagataaactgttaaggattgataatactttttggagaatgttcagcaaggttatgtttgaatcaaaggttaagaaaaggataatgttatatgatgtaaaatttctatcattagtaggttatatagggtttaagaattggtttatagaacagttgagatcagctgagttgcatgaggtaccttggattgtcaatgccgaaggtgatctggttgagatcaagtcaattaaaatctatttgcaactgatagagcagagtttatttttaagaataactgttttgaactatacagatatggcacatgctctcacaagattaatcagaaagaagttgatgtgtgataatgcactattaacttcaattccatccccaatggttaacttaactcaagttattgatcctacagaacaattagcttatttccctaagataacatttgaaaggctaaaaaattatgacactagttcaaattatgctaaaggaaagctaacaaggaattacatgatactgttgccatggcaacatgttaatagatataactttgtctttagttctactggatgtaaagttagtctaaaaacatgcattggaaaacttatgaaagatctaaaccctaaagttctgtactttattggagaaggggcaggaaattggatggccagaacagcatgtgaatatcctgacatcaaatttgtatacagaagtttaaaagatgaccttgatcatcattatcctttggaataccagagagtgataggagaattaagcaggataatagatagtggtgaagggctttcaatggaaacaacagatgcaactcaaaaaactcattgggatttgatacacagagtaagcaaagatgctttattaataactttatgtgatgcagaatttaaggacagagatgatttttttaagatggtaattctatggaggaaacatgtattatcatgcagaatttgcactacctatgggacagacctctatttattcgcaaagtatcatgctaaagactgcaatataaaattacctttttttgtgagatcagttgccacctttattatgcaaggtagtaaactgtcaggctcggaatgctacatactcttaacactaggccaccacaacaatttaccttgtcatggagaaatacaaaattctaagatgaaaatagcagcgtgtaatgatttttatgctgcaaaaaaacttgacaataaatcaattgaagccaactgtaaatcacttttatcagggctaagaataccgataaataagaaggaattaaatagacagagaaggttattaacactacaaagcaaccattcttctgtagcaacagttggaggtagcaaggtcatagagtctaaatggttaacaaacaaggcaaacacaataattgattggttagaacatattttaaattctccaaaaggtgaattaaattatgatttttttgaagcattagaaaatacttaccctaatatgattaaactaatagataatctagggaatgcagagattaaaaaactgatcaaagtaactggatatatgcttgtaagtaaaaaatgaaaaatgataaaaatgataaaataggtgacaacttcatactattccaaagtaatcatttgattatgcaattatgtaatagttaattaaaaactaaaaatcaaaagttaaaaactaataactgtcattaagtttattaaaaataagaaattataattggatgtatacggtttttttgccgt

SEQ ID NO: 6 - DNA, 13220 bp, Artificial Sequence
  GFP coding sequence is inserted between (40) and (784)
gcgaaaaaaacgcgtataaattagattacaaaaaaatatgggacaagtgaaaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagttaattaaaaagtgggacaagtgaaaatgtctcttcaagggattcacctgagtgatctatcatacaagcatgctatattaaaagagtctcagtacacaataaaaagagatgtgggtacaacaactgcagtgacaccctcatcattgcaacaagaaataacgctgttgtgtggagaaattctgtatgctaaacatgctgattacaaatatgctgcagaaataggaatacaatatattagcacagctttaggatcagagagagtgcagcagattctgaggaactcaggcagtgaagtccaagtggtcttaaccagaacgtactctctggggaaagttaaaaacaataaaggagaagatttacagatgttagacatacacggggtagagaagagctgggtagaagagatagacaaagaagcaaggaaaacaatggcaaccttgcttaaggaatcatcaggtaatatcccacaaaatcagaggccctcagcaccagacacacccataatcttattatgtgtaggtgcattaatatttactaagctagcatcaaccatagaagtgggactagagaccacagtcagaagggctaaccgtgtactaagtgatgcactcaagagataccctagaatggacatcccaaaaattgccagatccttctatgacttatttgaacaaaaagtgtatcacagaagtttgttcattgagtatggcaaagcattaggctcatcatctacaggcagcaaagcagaaagtctatttgttaatatattcatgcaagcttatggagccggtcaaacaatgctaaggtggggggtcattgccaggtcatccaacaatataatgttaggacatgtatctgtccaagctgagttaaaacaggtcacagaagtctatgacttggtgcgagaaatgggccctgaatctggacttctacatttaaggcaaagcccaaaagctggactgttatcactagccaactgtcccaactttgcaagtgttgttctcggaaatgcctcaggcttaggcataatcggtatgtatcgtgggagagtaccaaacacagaattattttcagcagcagaaagttatgccaaaagtttgaaagagagcaataaaatcaatttctcttcattaggacttacagatgaagagaaagaggctgcagaacatttcttaaatgtgagtgacgacagtcaaaatgattatgagtaattaaaaaagtgggacaagtcaaaatgtctttccctgaaggaaaagatattcttttcatgggtaatgaagcagcaaaattagcagaagctttccagaaatcattaaggaaaccaagtcataaaagatctcaatctattataggagaaaaagtgaacactgtatcagaaacattggaattacctactatcagtagacctgcaaaaccaaccatactgtcagaaccaaagttagcatggactgataaaggtggggcaatcaaaactgaaataaagcaagcaatcaaagtcatggatcctattgaggaagaagagtctactgagaagaaggtgctgccctccagtgatgggaaaacccctgcagaaaagaaactgaaaccatcaactaacaccaaaaagaaagtttcgtttacaccaaatgaaccaggaaaatatacaaagttggaaaaagatgctctagatttgctctcagataatgaagaagaagatgcagaatcttcaatcttaacctttgaagaaagagatacttcatcgttaagcattgaggccagattggaatcaatagaggagaaattaagcatgatattagggctattaagaacactcaacattgctacagcaggacccacagcagcaagagatgggatcagagatgcaatgattggcgtaagagaggaattaatagcagacataataaaggaagccaaagggaaagcagcagaaatgatggaagaggaaatgagtcaacgatcaaaaataggaaacggtagtgtaaaactaacagagaaagcaaaagagcttaacaaaattgttgaagatgaaagcacaagtggagaatctgaagaagaagaagaaccaaaagacatacaagacaatagtcaagaagatgacatttaccagttaattatgtagtttaataaaaataaacaatgggacaagtaaaaatggagtcctacctagtagacacttatcaaggcattccttacacagcagctgttcaagttgatctaatagaaaaggacctgttacctgcaagcctaacaatatggttccctttgtttcaggccaacacaccaccagcagtgctgctcgatcagttgaaaaccctaacaataaccactctgtatgctgcatcacaaaatggtccaatactcaaagtgaatgcatcagcccaaggtgcagcaatgtctgtacttcccaaaaaatttgaagtcaatgcgactgtagcactcgatgaatatagcaaattggaatttgacaaactcacagtctgtgaagtaaaaacagtttacttaacaaccatgaaaccatacgggatggtatcaaaatttgtgagctcagccaaatcagttggcaaaaaaacacatgatctaatcgcactgtgtgattttatggatctagaaaagaacacacctgttacaataccagcattcatcaaatcagtttcaatcaaagagagtgagtcagctactgttgaagctgctataagcagtgaagcagaccaagctctaacacaggccaaaattgcaccttatgcgggattgattatgatcatgactatgaacaatcccaaaggcatattcaaaaagcttggagctgggactcaagttatagtagaactaggagcatatgtccaggctgaaagcataagtaaaatatgcaagacttggagccatcaagggacaagatatgtgttgaagtccagataacagccaagcaccttggccaagagctactaactctatctcatagattataaagtcaccattctagttatataaaaatcaagttagaacaagaattaaatcaatcaagaatgggacaaataaaaatgtcttggaaagtggtgatcattttttcattgttaataacacctcaacacggtcttaaagagagctatttagaagagtcatgtagcactataactgaaggatatctcagtgttctgaggacaggttggtataccaacgtttttacactggaggtaggtgatgtagagaaccttacatgtgctgatggacctagcttaataaaaacagaattagacctgaccaaaagtgcactaagagaactcagaacagtttctgctgatcaactggcaagagaggagcaaattgagaatcccagacaatctagatttgttctaggagcaatagcactcggtgttgcaacagcagctgcagttacagcaggtgttgcaattgccaaaaccatccggcttgaaagtgaagtaacagcaattaagaatgccctcaaaaagaccaatgaagcagtatctacattggggaatggagttcgagtgttggcaactgcagtgagggagctggaagattttgtgagcaagaatctaacacgtgcaatcaacaaaaacaagtgcgacattgctgacctgaaaatggccgttagcttcagtcaattcaacagaaggtttctaaatgttgtgcggcaattttcagacaatgctggaataacaccagcaatatccttggacttaatgacagatgctgaactagccagagctgtttccaacatgccaacatctgcaggacaaataaaactgatgttggagaaccgtgcaatggtaagaagaaaggggttcggaatcctgataggagtttacggaagctccgtaatttacatggtgcaactgccaatctttggagttatagacacgccttgctggatagtaaaagcggccccttcttgctcagaaaaaaagggaaactatgcttgccttttaagagaagatcaaggatggtattgtcagaatgcagggtcaactgtttactacccaaatgaaaaagactgcgaaacaagaggagaccatgtcttttgcgacacagcagcaggaatcaatgttgctgagcagtcaaaggagtgcaacatcaacatatccactactaattacccatgcaaagttagcacaggaagacaccctatcagtatggttgcactgtctcctcttggggctttggttgcttgctacaagggagtgagctgttccattggcagcaacagagtagggatcatcaagcaactgaacaaaggctgctcttatataaccaaccaagacgcagacacagtgacaatagacaacactgtataccagctaagcaaagttgagggcgaacagcatgttataaaaggaaggccagtgtcaagcagctttgatccagtcaaatttcctgaagatcaattcaatgttgcacttgaccaagttttcgaaagcattgagaacagtcaggccttggtggatcaatcaaacagaatcctaagcagtgcagagaaaggaaacactggcttcatcattgtaataattctaattgctgtccttggctctaccatgatcctagtgagtgtttttatcataataaagaaaacaaagaaacccacaggagcacctccagagctgagtggtgtcacaaacaatggcttcataccacataattagttaattaaaaataaagtaaattaaattaaaataaaataaaattaaaattaaaataaaataaaaataaaaatttgggacaaatcataatgtctcgcaaggctccatgcaaatatgaagtgcggggcaaatgcaatagaggaagtgagtgcaagtttaaccacaattactggagttggccagatagatacttactaataagatcaaattatttattaaatcaacttttaaggaacactgatagagctgatggcttatcaataatatcaggagcaggcagagaagataggacacaagattttgtcctaggttccaccaatgtggttcaaggttatattgatgataaccaaagcataacaaaagctgcagcctgttacagtctacataatataatcaaacaactacaagaagttgaagttaggcaggctagagataacaaaccatctgacagcaaacatgtggcacttcacaacttagtcctatcttatatggagatgagcaaaattcctgcatctttaatcaacaatctcaaaagactgccgagagagaaactgaaaaaattagcaaagcttataattgacttatcagcaggtgctgaaaatgactcttcatatgccttgcaagacagtgaaagcactaatcaagtgcagtgagcatggtcctgttttcattactatagaggttgattacatgatatggactcataaggacttaaaagaagctttatctaatgggatagtgaagtctcatactaacatttacaattgttatttagaaaacatagaaattatatatgtcaaggcttacttaagttagtaaaaacacatcagagtgggataaatgacaatgataacattagatgtcattaaaagtgatgggtcttcaaaaacatgtactcacctcaaaaaattaattaaagaccactctggtaaagtgcttattgtacttaagttaatattagctttactaacatttctcacagtgacaatcaccatcaattatataaaagtagaaaacaatctgcaaatatgtcagtcaaaaactgaatcagacaaaaaggactcatcatcaaataccacatcagtcacaaccaagactactctaaatcatgatataacacagtattttaaaagtttgattcaaaggtatacaaactctgcaataaacagagacacatgctggaaaataagcagaaatcaatgcacaaacataacaacatacaaatttttatgttttaaatctgaagaaacaaaaaccaacaattgtgataaactgacagatttatgcagaaacaaaccaaaaccagctgttgaagtgtatcacatagtagaatgccattgtatatacacagttaaatggaagtgctatcattacccaatagatgaaacccaatcctaaataacactagattaggatccatccaagtctgttagttcaacaatttagttatttaaaaatattttgaaaacaagtaagtttctatgatacttcataataataagtaataattaattgcttaatcatcatcacaacattattcgaaaccataactattcaatttaagaagtaaaaacaataatatgagacaaataacaatggatcctcttaatgaatccactgttaatgtctatcttcccgactcatatcttaaaggagtgatttcttttagtgagactaatgcaattggttcatgtctcttaaaaagaccttacctaaaaaatgacaacactgcaaaagttgccatagagaatcctgttatcgagcatgttagactcaaaaatgcaatcaattctaagatgaaaatatcagattacaggatagtagagccagtaaacatgcaacatgaaattatgaagaatgtacacagttgtgagctcacattattaaaacagtttttaacaaggagtaaaaatattagcactcttaaattaaatatgatatgtgattggctgcagttaaagtctacatcagatgatacctcaatcttaagttttatagatgtagaatttatacctagctgggtaagcaattggtttagtaattggtacaatctcaacaagttgattctggaattcaggaaagaagaagtaataagaactggttcaatcttgtgtaggtcattgggtaaattagtttttgttgtatcatcatacggatgtatagtcaagagcaacaaaagcaaaagagtgagcttcttcacatacaatcaactgttaacatggaaagatgtgatgttaagtagattcaatgcaaatttctgtatatgggtaagcaacagtctgaatgaaaatcaagaagggttagggttgagaagtaatctgcaaggcatattaactaataagctatatgaaactgtagattatatgcttagtttgtgttgcaatgaaggtttctcacttgtgaaagagtttgagggttttattatgagtgaaatccttaggattactgaacatgctcaattcagtactagatttagaaatactttattaaatggattaactgatcaattgacaaaattaaaaaataaaaacagactcagagttcatggtaccgtgttagaaaataatgattatccaatgtatgaagttgtacttaaattattaggagatactttgagatgtattaaattattaatcaataaaaacttagagaatgctgctgaattatactatatatttagaatattcggtcacccaatggtagatgaaagagatgcaatggatgctgtcaaattaaacaatgaaatcacaaaaatcctcaggttggagagcttgacagaactaagaggggcattcatattaaggattatcaaaggatttgtagacaacaacaaaagatggccgaaaattaaaaacttaaaagtgcttagtaaaagatggactatgtacttcaaagcaaaaagttaccctagtcaacttgaattaagtgaacaagattttttagagcttgctgcaatacagtttgaacaagagttttctgttcctgaaaaaaccaaccttgagatggtattaaatgataaagctatatcacctcctaaaagattaatttggtctgtgtacccaaaaaattacttacctgagacaataaaaaatcgatatctagaagagactttcaatgcaagtgatagtctcaaaacaagaagagtactagagtactatttgaaagataataaattcgaccaaaaagaacttaaaagttatgtggttaaacaagaatatttaaatgataaggatcatattgtctcgctaactggaaaagaaagagaattaagtgtaggtagaatgtttgctatgcaaccaggaaaacagcgacaaatacaaatattggctgaaaaattgttagctgataatattgtaccctttttcccagaaactttaacaaagtatggtgatctagatcttcagagaataatggaaatcaaatcagaactttcttctattaaaaccagaagaaatgatagttataataattacattgcaagagcatccatagtaacagatttaagtaagttcaaccaagcctttaggtatgaaactacagcgatctgtgcggatgtagcagatgaactacatggaacacaaagcctattctgttggttacatcttatcgttcctatgactacaatgatatgtgcctatagacatgcaccaccagaaacaaaaggtgaatatgatatagataagatagaagagcaaagtggtttatatagatatcatatgggtggtattgaaggatggtgtcaaaaactctggacaatggaagctatatctttattagatgttgtatctgtaaagacacgatgtcaaatgacatctttattaaacggtgacaaccaatcaatagatgtaagtaaaccagttaagttatctgagggtttagatgaagtgaaagcagattatagcttggctgtaaaaatgctaaaagaaataagagatgcatacagaaatataggccataaacttaaagaaggggaaacatatatatcaagagatcttcagtttataagtaaggtgattcaatctgaaggagtaatgcatcctacccctataaaaaagatcttaagagtgggaccatggataaacacaatattagatgacattaaaaccagtgcagagtcaatagggagtctatgtcaggaattagaatttaggggggaaagcataatagttagtctgatattaaggaatttttggctgtataatttatacatgcatgaatcaaagcaacaccccctagcagggaagcagttattcaaacaactaaataaaacattaacatcagtgcagagattttttgaaattaaaaaggaaaatgaagtagtagatctatggatgaacataccaatgcagtttggaggaggagatccagtagtcttctatagatctttctatagaaggacccctgattttttaactgaagcaatcagtcatgtagatattctgttaaaaatatcagccaacataagaaatgaagcgaaaataagtttcttcaaagccttactgtcaatagaaaaaaatgaacgtgctacactgacaacactaatgagagaccctcaagctgtgggctcagagcgacaagcaaaagtaacaagtgatatcaatagaacagcagttaccagcatcttaagtctttctccaaatcaacttttcagcgatagtgctatacactacagtagaaatgaagaagaggtcggaatcattgctgacaacataacacctgtttatcctcatggactgagagttttgtatgaatcattaccttttcataaagctgaaaaagttgtaaatatgatatcaggaacaaaatccataaccaacttattacagagaacatctgctattaatggtgaagatattgacagagctgtatccatgatgctggagaacctaggattattatctagaatattgtcagtagttgttgatagtatagaaattccaaccaaatctaatggtaggctgatatgttgtcagatatctagaaccctaagggagacatcatggaataatatggaaatagttggagtaacatcccctagcatcactacatgcatggatgtcatatatgcaactagctctcatttgaaagggataatcattgaaaagttcagcactgacagaactacaagaggtcaaagaggtccaaagagcccttgggtaggatcgagcacgcaagagaaaaaattagttcctgtttataacagacaaattctttcaaaacaacaaagagaacagctagaagcaattggaaaaatgagatgggtatataaagggacaccaggtttaagacgattactcaataagatttgtcttggaagtttaggcattagttacaaatgtgtaaaacctttattacctaggtttatgagtgtaaatttcctacacaggttatctgtcagtagtagacctatggaattcccagcatcagttccagcttatagaacaacaaattaccattttgacactagtcctattaatcaagcactaagtgaaagatttgggaatgaagatattaatttggtcttccaaaatgcaatcagctgtggaattagcataatgagtgtagtagaacaattaactggtaggagtccaaaacagttagttttaataccccaattagaagaaatagacattatgccaccaccagtgtttcaagggaaattcaattataagctagtagataagataacttctgaccaacatatcttcagtccagacaaaatagatatgttaacactggggaaaatgctcatgccaactataaaaggtcagaaaacagatcagttcttgaacaagagagagaattatttccatgggaacaatcttattgagtctttgtcagcagcgttagcatgtcattggtgtgggatattaacagagcaatgtatagaaaataatattttcaagaaagactggggtgacgggttcatatcggatcatgcttttatggacttcaaaatattcctatgtgtctttaaaactaaacttttatgtagttggggatcccaagggaaaaacattaaagatgaagatatagtagatgaatcgatagataaactgttaaggattgataatactttttggagaatgttcagcaaggttatgtttgaatcaaaggttaagaaaaggataatgttatatgatgtaaaatttctatcattagtaggttatatagggtttaagaattggtttatagaacagttgagatcagctgagttgcatgaggtaccttggattgtcaatgccgaaggtgatctggttgagatcaagtcaattaaaatctatttgcaactgatagagcagagtttatttttaagaataactgttttgaactatacagatatggcacatgctctcacaagattaatcagaaagaagttgatgtgtgataatgcactattaacttcaattccatccccaatggttaacttaactcaagttattgatcctacagaacaattagcttatttccctaagataacatttgaaaggctaaaaaattatgacactagttcaaattatgctaaaggaaagctaacaaggaattacatgatactgttgccatggcaacatgttaatagatataactttgtctttagttctactggatgtaaagttagtctaaaaacatgcattggaaaacttatgaaagatctaaaccctaaagttctgtactttattggagaaggggcaggaaattggatggccagaacagcatgtgaatatcctgacatcaaatttgtatacagaagtttaaaagatgaccttgatcatcattatcctttggaataccagagagtgataggagaattaagcaggataatagatagtggtgaagggctttcaatggaaacaacagatgcaactcaaaaaactcattgggatttgatacacagagtaagcaaagatgctttattaataactttatgtgatgcagaatttaaggacagagatgatttttttaagatggtaattctatggaggaaacatgtattatcatgcagaatttgcactacctatgggacagacctctatttattcgcaaagtatcatgctaaagactgcaatataaaattacctttttttgtgagatcagttgccacctttattatgcaaggtagtaaactgtcaggctcggaatgctacatactcttaacactaggccaccacaacaatttaccttgtcatggagaaatacaaaattctaagatgaaaatagcagcgtgtaatgatttttatgctgcaaaaaaacttgacaataaatcaattgaagccaactgtaaatcacttttatcagggctaagaataccgataaataagaaggaattaaatagacagagaaggttattaacactacaaagcaaccattcttctgtagcaacagttggaggtagcaaggtcatagagtctaaatggttaacaaacaaggcaaacacaataattgattggttagaacatattttaaattctccaaaaggtgaattaaattatgatttttttgaagcattagaaaatacttaccctaatatgattaaactaatagataatctagggaatgcagagattaaaaaactgatcaaagtaactggatatatgcttgtaagtaaaaaatgaaaaatgataaaaatgataaaataggtgacaacttcatactattccaaagtaatcatttgattatgcaattatgtaatagttaattaaaaactaaaaatcaaaagttaaaaactaataactgtcattaagtttattaaaaataagaaattataattggatgtatacggtttttttgccgt

(19) United States
(12) Patent Application Publication
(10) Pub. No.: US 2022/0054618 A1
(43) Pub. Date: Feb. 24, 2022

(54) PRODUCTION OF VIRAL VACCINES ON AN AVIAN CELL LINE

(71) Applicants: UNIVERSITE CLAUDE BERNARD LYON 1, VILLEURBANNE (FR); CENTRE NATIONAL DE LA RECHERCHE SCIENTIFIQUE (CNRS), Paris (FR); INSTITUT NATIONAL DE LA SANTE ET DE LA RECHERCHE MEDICALE (INSERM), Paris (FR); UNIVERSITE LAVAL, QUEBEC (CA); TRANSGENE, ILLKIRCH GRAFFENSTADEN (FR); ECOLE NORMALE SUPERIEURE DE LYON, Lyon (FR)

(72) Inventors: Manuel ROSA-CALATRAVA, Lyon (FR); Guy BOIVIN, Quebec (CA); Julia DUBOIS, RILLIEUX-LA-PAPE (FR); Mario Andres PIZZORNO, Lyon (FR); Olivier TERRIER, Lyon (FR); Aurélien TRAVERSIER, VENISSIEUX (FR)

(21) Appl. No.: 17/413,228

(22) PCT Filed: Dec. 12, 2019
(86) PCT No.: PCT/FR2019/053036

§ 371 Date: Jun. 11, 2021

(30) Foreign Application Priority Data
Dec. 14, 2018 (FR) 1872957

(51) Int. Cl. A61K 39/12; C12N 7/00; A61P 31/12

(52) U.S. Cl. CPC A61K 39/12; C12N 7/00; A61K 2039/5254; C12N 2760/18334; C12N 2760/18351; A61P 31/12

(57) ABSTRACT

The present invention relates to the use of the immortalised cell line ECACC 09070703, deposited on 7 Jul. 2009 at the European Collection of Authenticated Cell Cultures (ECACC, Salisbury, UK) under the number 09070703, for the production of a viral vaccine constituted of an attenuated strain derived from a human metapneumovirus.

### FIELD OF THE INVENTION

The present invention relates to the use of an immortalised avian cell line for the production of a pneumovirus type virus, and viral vaccines constituted of live attenuated viral strains.

### PRIOR ART

Pneumoviruses

Pneumoviruses are viruses responsible for acute respiratory tract infections such as bronchiolitis, bronchitis or pneumonia, mainly in populations at risk which are young children less than 5 years old, the elderly and immunodeficient persons.

The Pneumoviridae family, the members of which were previously included in the Paramyxoviridae family, comprises enveloped viruses with a single RNA strand of negative polarity, which include:
the human respiratory syncytial virus (hRSV), representing the orthopneumovirus sub-family, and
the human metapneumovirus (hMPV), representing the metapneumovirus sub-family (according to the International Committee on Taxonomy of Viruses (ICTV)).

At present, there is no vaccine available on the market, neither any specific and effective treatment, against these hMPV and/or hRSV viruses.

hRSV is the most frequent cause of respiratory infections in young children. Highly contagious, this virus mainly infects infants less than two years old.

hMPV is also one of the major causes of paediatric bronchiolitis, responsible for 5 to 15% of hospitalisations imputable to acute infections of the lower respiratory tracts in young children. The average age of children hospitalised following an infection by hMPV is from 6 to 12 months, i.e. later than that caused by hRSV, which mainly arises between 0 and 3 months.

Both viruses are also the etiological agents responsible for 12 to 15% of consultations for infections of the upper and lower respiratory tracts of non-hospitalised children.

In terms of treatment, ribavirin, not exempt of adverse effects, or instead intravenous immunoglobulins, very expensive, may be used occasionally for the treatment of serious cases of infections by hMPV, as with infections by hRSV.

Other types of treatments are under development and/or in the course of characterisation such as fusion inhibitor peptides, sulphated glycosaminoglycans, RNA inhibitors and certain immunomodulators.

The usual clinical approach, widely favoured today, consists in treating essentially the symptoms of the infection, by placing patients under respiratory assistance (administration of oxygen or mechanised ventilation) and by administering to them bronchodilators, corticosteroids and/or antibiotics to prevent and/or treat secondary bacterial infections.

### Vaccination Strategies Developed for the Prevention of Pneumovirus Infections

With regard to vaccination, since the main populations targeted by hMPV are infants, young children and the elderly, it is crucial to dispose of effective and safe vaccines, in order to reduce severe respiratory diseases which have a dramatic impact in these age ranges.

The development of vaccines against pneumoviruses such as hMPV and/or hRSV thus represents not only a major health challenge, but also a real socio-economic issue with the objective of (i) reducing the important costs of treatments and hospitalisations associated with these infections, and (ii) decreasing the use of antibiotics in the context of secondary bacterial infections, and thus limiting the emergence of resistance.

Different vaccine strategies have been developed to date (Mazur et al., 2018).

For example, the Novavax Company has developed a “non-living, nanoparticle” vaccine candidate. This vaccine is composed of nanoparticles presenting a F protein from hRSV, genetically modified in order to increase its immunogenicity. This vaccine candidate is intended for pregnant women, it could thus be used to generate transient immunisation of the unborn child in utero.

The GSK Company is also developing a vaccine intended for pregnant women to immunise the foetus in utero, based on recombinant antigens derived from the F protein from hRSV.

Another approach consists in using non-pathogenic viral vectors, such as adenoviruses, by making them express pneumovirus antigens. A vaccine candidate intended for the new born, composed of an adenovirus coding for 3 major pneumovirus antigens (F, N and M2.1 proteins) is currently under development by the GSK Company.

These vaccine approaches are based on the injection or the expression of recombinant proteins, in different forms. Yet, the drawback of these approaches is the low immunogenicity inherent to these proteins, which by nature only induce a slight immune response. The addition of potentially harmful adjuvants, such as aluminium salts, must thus be envisaged in most cases.

Consequently, approaches based on the use of so-called “live attenuated” vaccines, constituted of attenuated viral strains, are to be favoured. Indeed, live attenuated vaccines have numerous advantages:
they may be administered by intra-nasal route and mimic the natural entry route of wild-type viruses, thus inducing an immune response quite similar to that physiologically observed after hMPV and/or hRSV infection;
it is a strategy that has never been described as being associated with an exaggerated inflammatory reaction (as may be observed after administration of inactivated vaccines);
this vaccine strategy does not require the addition of adjuvants, the live attenuated vaccine being by nature sufficiently immunogenic to generate a satisfactory immune reaction.

These approaches are particularly interesting for the vaccination of children, from 6 months old.

### Difficulties Linked to the Preparation of Live Attenuated Viral Vaccines on an Industrial Scale

These viral vaccines are produced by a step of viral replication on host cells of the virus, cultured in vitro. The choice of these cells is primordial: they must be at one and the same time:

(i) host cells of said virus, that is to say permissive vis-à-vis infection by the virus and replication of said virus, and

(ii) “industrialisable” cells, that is to say complying with the regulations in force for the production of vaccines on the industrial scale.

To date, there is no registered industrial cell line which has capacities of permissivity and production of live attenuated vaccine candidates against pneumovirus infections.

Laboratory cell lines, in their “adherent” form in culture, such as:
the MDCK cell line, a canine cell line,
the Vero cell line, an African green monkey kidney cell line, commonly used in cell culture for testing various viruses,
the PERC6 cell line, a cell line of human origin, and
the LLC-MK2 cell line, a cell line derived from Rhesus monkey kidney cells, available at the ATCC under the number CCL-7, and commonly used for tests of infection by various viruses,
   have been used for the experimental development of certain vaccine candidates (see tables 1 to 3); however, these adherent cell lines did not have the characteristics necessary to be used on an industrial scale.

For the production of vaccines having viral type replication, “industrialisable” cell lines, that is to say stable (“robust”), non-adherent, being able to be cultured in the absence of serum (for example), and complying with regulatory requirements for the production of viral vaccines intended to be administered to human beings or to animals, have been established.

Two types of cell lines are notably conventionally used:
The EB66® cell line developed by VALNEVA is a line derived from duck embryonic cells; it has already enabled the development of several viral vaccines.
The AGE1.CR® cell lines distributed by ProBioGen are derived from several cell types, from gallinacea or humans. In particular, the AGE1.CR.pIX® line is derived from Muscovy duck cells (Jordan et al., 2009). This very stable line enables the production, on an industrial scale, of vectors derived from alphavirus and paramyxovirus genomes, and for the growth of poxviruses.

However, to date, the production of hRSVs and hMPVs has not been able to be carried out on these established cell lines, well known to those skilled in the art.

The present invention pertains to the identification of an industrialisable immortalised duck cell line enabling the replication of viral vectors derived from hMPV and/or hRSV, notably the replication of live attenuated viral vaccines derived from a specific hMPV viral strain.

### DESCRIPTION OF THE INVENTION

At the present time, no vaccines exist making it possible to prevent infections by pneumoviruses, responsible for acute infections of the respiratory tracts. This is in part due to the fact that the reproduction of these viruses in vitro is difficult. In particular, these viruses have not been described as being able to multiply on known and well established industrialisable cell lines, such as the EB66® and AGE1.CR® lines.

The present invention pertains to the identification of an “industrialisable” cell line, which enables the replication of live attenuated viral vaccines intended to prevent infections by hMPV and/or hRSV.

More specifically, the present invention relates to the use of the immortalised cell line ECACC 09070703, deposited at the European Collection of Authenticated Cell Cultures (ECACC, Salisbury, UK) under the number 09070703 on 7 Jul. 2009, for the production of a viral vaccine constituted of an attenuated strain derived from a human metapneumovirus.

Said viral vaccine could notably be an attenuated strain derived from a human metapneumovirus comprising the genome sequence represented by the sequence SEQ ID NO. 1.

According to a particular embodiment, said viral vaccine is an attenuated viral strain that has been genetically modified by introduction of at least one exogenous gene, notably a gene coding for an antigen derived from hRSV, such as the F fusion protein for example.

The present invention also pertains to a method for producing a viral vaccine constituted of an attenuated strain derived from a human metapneumovirus, comprising the following steps:
a) Infection of cells in culture of the line deposited at the ECACC under the access number 09070703, by an attenuated viral strain derived from a human metapneumovirus;
b) Culture of said cells infected at step (a) for a duration comprised between 2 and 14 days, in a suitable medium;
c) Harvesting of the viral vaccine constituted of infectious viral particles of said attenuated viral strain produced during step (b).

The present invention also relates to a viral vaccine such as obtained by the method described above, as well as a pharmaceutical composition comprising said viral vaccine, and at least one pharmaceutically acceptable vehicle.

According to another aspect, the present invention pertains to said viral vaccine or to said pharmaceutical composition, for their use as medicine.

More specifically, the present invention pertains to said viral vaccine or to said pharmaceutical composition, for their use in the prevention or the treatment of viral infections, notably infections by pneumoviruses, and more particularly by human metapneumovirus and/or human respiratory syncytial virus.

The present invention also relates to a kit for the implementation of the method for producing a viral vaccine, comprising at least:
The immortalised cell line ECACC 09070703; and
An attenuated viral strain derived from a human metapneumovirus comprising the genome sequence represented by the sequence SEQ ID NO. 1, and in particular an attenuated viral strain comprising one of the genome sequences represented in SEQ ID NO. 2 or NO.3.

### DESCRIPTION OF THE FIGURES

FIG. 1. Replicative capacity of the wild viral strain C-85473 in DuckCelt®-T17 cells, in comparison with the replicative capacities of the wild strains CAN98-75, CAN97-82 and CAN99-81. The kinetics are stopped (*) when more than 50% of the cells are dead.
FIG. 1a Monitoring of the amount of cells as a function of post viral-infection days
[FIG. 1b] Monitoring of the viral load (TCID50/ml) as a function of post viral-infection days

FIG. 2. Replicative capacities of the wild recombinant C-85473 WT (GFP) and attenuated ΔSH-C-85473 (GFP) and ΔG-C-85473 (GFP) viruses in DuckCelt®-T17 cells.
FIG. 2a Cell growth after infection. The Mock cells were not infected.
FIG. 2b Infectiveness of the recombinant viruses C-85473 WT (GFP), ΔSH-C-85473 (GFP) and ΔG-C-85473 (GFP). The percentage infected cells is evaluated by flow cytometry (detection of GFP expression expressed by these recombinant viruses) during the 14 days of viral kinetics.
FIG. 2c Viral replication of the recombinant viruses C-85473 WT (GFP), ΔSH-C-85473 (GFP) and ΔG-C-85473 (GFP). Viral production is measured by assaying the number of infectious particles per ml of culture medium (expressed in TCID₅₀/ml) from samples taken every 2 to 3 days during the 14 days of viral kinetics.

FIG. 3. Replicative capacities in LLC-MK2 cells and in a 3D reconstituted human respiratory epithelium model and cultured at the air-liquid interface (MucilAir™) of the recombinant viruses ΔSH-C-85473 (GFP) and ΔG-C-85473 (GFP) produced in DuckCelt®-T17 cells.
FIG. 3a A cell lawn of LLC-MK2 cells (on the left) and MucilAir™ healthy reconstituted human respiratory epitheliums (on the right) were infected by the recombinant hMPV ΔSH-C-85473 at MOI (multiplicity of infection) of 0.01 and 0.1, respectively. The photos were taken after 3, 5, 7, 12 and 17 days post-infection for the respiratory epithelium.
FIG. 3b A cell lawn of LLC-MK2 cells (on the left) and MucilAir™ healthy reconstituted human respiratory epitheliums (on the right) were infected by the recombinant hMPV ΔG-C-85473 at MOI (multiplicity of infection) of 0.1 and 0.65, respectively. The photos were taken after 3, 5, 7, 12 and 17 days post-infection for the respiratory epithelium.
FIG. 3c Viral secretion at the apical pole of infected epitheliums was evaluated by RT-qPCR (number of copies of the N viral gene) from apical surface washings carried out at 5, 7, 12 and 17 days post-infection. The viral replication of the attenuated recombinant viruses ΔSH-C-85473 and ΔG-C-85473 was compared to that of a wild recombinant virus C-85473 produced in DuckCelt®-T17 cells. The detection threshold by RT-qPCR is 10 copies of N viral gene.
FIG. 3d Viral replication within infected epitheliums was evaluated by RT-qPCR (number of copies of the N viral gene) from epithelium lysates at the 17th day of post-infection. The viral replication of the attenuated recombinant viruses ΔSH-C-85473 and ΔG-C-85473 was compared to that of a wild recombinant virus WT C-85473 produced in DuckCelt®-T17 cells. The detection threshold by RT-qPCR is 10¹ copies of N viral gene.

FIG. 4. The recombinant viruses ΔSH-C-85473 (GFP) and ΔG-C-85473 (GFP) produced on DuckCelt®-T17 cells conserve their in vivo attenuation character.
[FIG. 4] BALB/c mice 4 to 6 weeks old were infected by intranasal route with 5×10⁵ TCID₅₀ of the vaccine candidates ΔSH-C-85473 or ΔG-C-85473 produced on DuckCelt®-T17 cells, or the culture medium (mock) as control. Daily monitoring of the weight and the mortality of the infected mice was carried out for 9 days after infection and compared with non-infected mice (mock).

### DETAILED DESCRIPTION OF THE INVENTION

The present invention relates to the use of the immortalised cell line ECACC 09070703, deposited on 7 Jul. 2009 at the European Collection of Authenticated Cell Cultures (ECACC, Salisbury, UK) under the number 09070703, for the production of a viral vaccine constituted of an attenuated viral strain derived from a human metapneumovirus.

ECACC Cell Line No 09070703

Thus, the present invention relates to a novel use of the DuckCelt®-T17 cell line, deposited at the European Collection of Authenticated Cell Cultures (ECACC) under the access number 09070703, and described previously in the literature, notably in the international applications WO 2007/077256, WO 2009/004016 and WO 2012/001075.

These applications pertain to the preparation of immortalised avian cell lines, and to the use thereof for virus reproduction.

The term “immortalised cell line” designates cells capable of growing in culture in vitro during at least 35 subcultures (dilution of the cells in a new culture medium), without losing their functional characteristics.

Briefly, the cell lines described in WO 2012/001075, deposited at the ECACC under the numbers 09070701, 09070702 and 09070703 are derived from embryonic duck (Cairina moschata) cells, and were immortalised by introduction of the following nucleotide sequences:
the nucleotide sequence of the E1A region derived from the genome of an adenovirus, coding for RNA 12S and 13S; and
the gene coding for duck telomerase reverse transcriptase (dTERT).

The main destination of these cell lines is their use for the replication of viruses, such as poxvirus, adenovirus, retrovirus, herpes virus and influenza virus.

Recently, Petiot and his collaborators (Petiot et al., 2018) have demonstrated the fact that the avian cell line DuckCelt®-T17, deposited at the ECACC under the number 09070703, could be advantageously used for the production of infectious particles of influenza virus of different origins (human, avian, porcine).

Attenuated Viral Strains of Human Metapneumovirus

Considerable research is currently being undertaken with the aim of producing a viral vaccine making it possible to prevent and/or treat infections by metapneumovirus and/or human respiratory syncytial virus.

In particular, attenuated viral strains derived from human metapneumovirus have been prepared and proposed as vaccines.

The genetic analysis of clinical strains of hMPV has made it possible to define two major groups (genotypes A and B) and four “minor” sub-groups (A1, A2, B1 and B2), mainly based on the sequence variability of attachment surface (G) and fusion (F) glycoproteins. It was next shown that these groups could further be sub-divided into sub-lines such as A2a, A2b and A2c (Huck et al., 2006; Nidaira et al., 2012).

Among widely studied viral strains may be cited the strain NL 00-1, belonging to the genotype A1; the strain CAN 97-83 belonging to the genotype A2; and the strain CAN 98-75, belonging to the genotype B2.

In the present application, the terms “virus” and “viral strain” are used indiscriminately to designate a particular viral strain, such as identified previously.

In the sense of the invention, “derived strain” is taken to mean a recombinant viral strain obtained by the introduction of genetic modifications in the genome of a so-called “original” viral strain. The original strain is advantageously a wild strain, for example a clinical isolate.

The virulence of a viral strain corresponds to the degree of rapidity of multiplication of a virus in a given organism, thus its invasion speed. “To attenuate the virulence” is thus taken to mean to decrease the invasion speed of a virus in an organism.

This attenuation may take the form of a decrease in the replication capacities of the viral strain, a decrease in the infection capacity of the target cells, or instead a decrease in the pathology induced by the viral infection of the organism.

Viral strains are considered as being attenuated in vitro when they exhibit decreased replicative capacity compared to the wild virus (WT), and/or when these viral strains lead to the more restricted formation of infectious outbreaks, notably of syncytia (adjacent cells fusing following viral infection). In vivo, the attenuated viral strains replicate at a lower maximum load and/or induce a less severe pathology (in terms of weight loss or inflammatory profile or histopathological disorders) than the wild virus strain.

Thus, “attenuated viral strain” is taken to mean, in the sense of the invention, a recombinant virus, the virulence of which is decreased compared to that of the original viral strain, that is to say less than that of the original viral strain.

To measure the virulence of a viral strain, in vitro, ex vivo or in vivo tests may be carried out, such as for example in vitro replicative capacity tests (measured by TCID₅₀/ml viral assay or quantification of viral genome by quantitative PCR), monitoring by microscopic observation of the evolution of in vitro and ex vivo cytopathic effects (in a 3D reconstituted human respiratory model and cultivated at the air-liquid interface for example), or monitoring of the clinical signs of the pathology and the measurement of pulmonary viral loads in an in vivo infection model.

Tables 1, 2 and 3 below list the different approaches under development to obtain live attenuated vaccines from wild hMPV viral strains.

TABLE 1
List of vaccine candidates attenuated on the basis of a hMPV strain having
one or more mutations, developed or under development

| | | | | Protective | |
|---|---|---|---|---|---|
| Name of the | | Attenuation | Attenuation | neutralising | |
| virus | Description | in vitro | in vivo | antibodies | Reference |
| cp-HMPV M11 | Insertion of 11 aa | ✓ Vero > 39° C. | ✓ H | ✓ H | (Herfst, de |
| (B1/NL/1/99) | mutations among the 17 | | | | Graaf et al. |
| | induced by passages on | | | | 2008) |
| | Vero cells, temperature | | | | |
| | decreasing down to | | | | |
| | 25° C. | | | | |
| cp-HMPV | 4 cp (cold passaging) | ✓ Vero > | ✓ H | ✓ H | (Herfst, de |
| HRSV3 | mutations of hRSV | 37° C. | | | Graaf et al. |
| (B1/NL/1/99) | | | | | 2008) |
| rhMPV-R329K | Mutations in the integrin | ✓ LLC-MK2 | ✓ CR | ✓ CR | (Wei, |
| rhMPV-D331A | binding domain of the F | | | | Zhang et |
| (A1/NL/1/00) | protein | | | | al. 2014) |
| HMPV M2 | Deletion of the N- | ✓ Vero. | ✓ M | ✓ M | (Yu, Li et |
| (A1/NL/1/00) | glycosylation site (aa | | ✓ M SCID | | al. 2012) |
| | 172) of the F protein | | | | (Liu, Shu et |
| | | | | | al. 2013) |
| HMPV-MTase | Mutations of the | ✓ LLC-MK2 | ✓ CR | ✓ CR | (Zhang, |
| (A1/NL/1/00) | methyltransferase site of | | | | Wei et al. |
| | L polymerase | | | | 2014) |
| | G1696A, G1700A, and | | | | |
| | D1755A | | | | |

The symbol ✓ indicates that the cells used are liable/permissive to viral infection by said viral strain.

The following abbreviations are used for the in vivo study models: M for Mouse; H for Hamster; CR for Cotton Rat; CM for Cynomolgus Macaque; AGM for African Green Monkey; Ch for Chimpanzee; Rh for Rhesus Monkey; and SCID for Severe Combined ImmunoDeficiency.

The abbreviation “aa” designates amino acids, “aa172” designating the amino acid in position 172 in the protein sequence.

Point mutations are represented according to the nomenclature known to those skilled in the art.

TABLE 2
List of live attenuated vaccine candidates developed or under development,
comprising a hMPV strain having complete deletion of at least one gene

| Name of the | | | | | |
|---|---|---|---|---|---|
| virus | | | | Protective | |
| (group/name of | | Attenuation | Attenuation | neutralising | |
| wild strain) | Description | in vitro | in vivo | antibodies | References |
| HMPV-ΔSH | Deletion of the | Ø LLC-MK2 | Ø H | ✓ H | (Biacchesi, |
| (A2/CAN97-83) | SH gene | | ✓ Ch | ✓ Ch | Skiadopoulos et |
| | | | | | al. 2004) |
| | | | | | (Biacchesi, Pham |
| | | | | | et al. 2005) |
| HMPV-ΔG | Deletion of the G | Ø LLC-MK2 | ✓ H at 3 days | ✓ H | (Biacchesi, |
| (A2/CAN97-83) | gene | | 0 H > 3 days | | Skiadopoulos et |
| | | | ✓ Ch | ✓ Ch | al. 2004) |
| | | | | | (Biacchesi, Pham |
| | | | | | et al. 2005) |
| HMPV-ΔSH/ΔG | Deletion of the | Ø LLC-MK2 | ✓ H at 3 | ✓ H | (Biacchesi, |
| (A2/CAN97-83) | SH and G genes | | days. | | Skiadopoulos et |
| | | | Ø H > 3 days | | al. 2004) |
| HMPV-ΔM2-2 | Mutations codon | Ø Vero | ✓ H | ✓ H | (Buchholz, |
| (A2/CAN97-83) | initiation + | | ✓ Ch | ✓ Ch | Biacchesi et al. |
| | codon stop | | | | 2005) (Biacchesi, |
| | | | | | Pham et al. 2005) |
| | | | | | (Schickli, Kaur et |
| | | | | | al. 2008) |
| HMPV-ΔM2-1 | Mutation codon | Ø Vero | Ø H | Ø H | (Buchholz, |
| (A2/CAN97-83) | stop | | | | Biacchesi et al. |
| | | | | | 2005) |
| HMPV-ΔM2-1/ | Deletion of the | Ø Vero | Ø H | Ø H | (Buchholz, |
| ΔM2-2 | complete M2 | | | | Biacchesi et al. |
| (A2/CAN97-83) | gene | | | | 2005) |

All the attenuated viral strains developed and described in this table 2 are derived from the wild strain CAN97-83 of the sub-group A2.

The following abbreviations are used:

Δ: total deletion of the gene. Ø=no attenuation or more replicative.

TABLE 3
List of live attenuated vaccine candidates developed or under development,
based on a chimeric hMPV strain

| | | | | Protective | | |
|---|---|---|---|---|---|---|
| Name of | | Attenuation | Attenuation | neutralising | Clinical | |
| the virus | Description | in vitro | in vivo | antibodies | test | Ref. |
| HMPV-Pa | hMPV genetic | ∅ Vero | ✓ H | ✓ H | Phase 1 | (Pham, |
| (A2/CAN97- | background (SH | | ✓ AGM | ✓ AGM | NCT01255410 | Biacchesi et |
| 83) | stabilised) | | | | | al. 2005) |
| | Exchange of the P | | | | | (Karron, San |
| | gene with the | | | | | Mateo et al. |
| | homologue | | | | | 2017) |
| | aMPV C | | | | | |
| HMPV Na | hMPV genetic | ∅ Vero | ✓ H at 3 | ✓ H | | (Pham, |
| (A2/CAN97- | background (SH | | days. | ✓ AGM | | Biacchesi et |
| 83) | stabilised) | | ∅ H > 3 | | | al. 2005) |
| | Exchange of the N | | days | | | |
| | gene with the | | ✓ AGM | | | |
| | homologue | | | | | |
| | aMPV C | | | | | |
| b/hPIV3/ | Bovine PIV-3 | — | ∅ H | ✓ H | | (Tang, |
| hMPV F2 | genetic | | ∅ AGM | ✓ AGM | | Schickli et al. |
| (A1/NL/ | background | | ✓ Rh | (hMPV/ | | 2003) |
| 1/100) | Exchange of the F | | seronegative | hPIV) | | (Tang, |
| | and HN genes | | | | | Mahmood et. |
| | with their | | | | | al. 2005) |
| | homologue | | | | | |
| | hPIV-3 | | | | | |
| | Addition F hMPV | | | | | |
| | in 2^{nd} position on | | | | | |
| | the genome | | | | | |
| SeV-MPV- | SeV genetic | — | ✓ CR | ✓ CR | | (Russell, |
| Ft | background | | | | | Jones et al. |
| (A2/CAN00- | Addition F gene | | | | | 2017) |
| 16) | hMPV truncated | | | | | |
| | for its TM domain | | | | | |

The following abbreviations are used:

TM=Transmembrane domain; PIV=Parainfluenza virus; SeV=Sendai virus.

Other attenuated strains derived from hMPV have been described in the applications or patents summarised below.

International application WO 2005/014626 pertains to several hMPV strains, designated as follows: CAN 97-83, CAN 98-75 and HMPV 00-1. This application describes a strain of recombinant hMPV, designated CAN 97-83, genetically modified to attenuate the virulence thereof. The proposed modifications notably concern the total deletion of genes coding for G and/or SH proteins.

U.S. Pat. No. 8,841,433 further describes other isolated hMPV strains and the use thereof for the preparation of vaccines.

In patent application FR1856801, attenuated strains derived from the clinical strain C-85473 of human metapneumovirus, comprising the genome sequence represented by the sequence SEQ ID NO. 1, have been described and proposed as vaccine candidates. These attenuated strains comprise one or more genetic modifications of said sequence SEQ ID NO.1, notably the inactivation of the gene coding for the SH protein and/or the gene coding for the G protein of said metapneumovirus strain.

All of these attenuated viral strains derived from human metapneumovirus are vaccine candidates that are capable of being developed industrially to produce, on a large scale, vaccines intended to be administered to numerous patients, with a preventive and/or therapeutic aim.

However, to attain this objective, it is necessary to identify a cell support that will enable the identified attenuated viral strains to be replicated on an industrial scale.

Tests carried out on industrial cell lines, such as EB66® and AGE1.CR.pIX® lines, did not make it possible to obtain sufficient replication of different viral strains derived from human metapneumovirus.

The present invention aims to respond to this need, having identified an immortalised cell line, having functional characteristics suitable to virus production on the industrial scale, for the replication of such attenuated strains derived from human metapneumovirus.

According to an embodiment of the invention, said attenuated strain has been genetically modified by inactivation of the gene coding for the SH protein and/or the gene coding for the G protein of metapneumovirus.

Genetic modifications designate, in the sense of the invention, all modifications of an original nucleotide sequence such as the deletion of one or more nucleotides, the addition of one or more nucleotides, and the replacement of one or more nucleotides. These modifications notably comprise all modifications making it possible to shift the genetic reading frame, or to introduce a codon stop in the middle of a coding sequence.

Among genetic modifications intended to attenuate the virulence of a virus strain, genetic modifications are notably known making it possible to inactivate or even to delete one or more genes coding for proteins non-essential for the growth of the virus in culture.

In the sense of the invention, the inactivation of a gene designates the fact that this gene is modified in such a way that the product of the gene is no longer expressed, expressed in non-active form, or expressed in a so small amount that the activity of this protein is inexistant. This inactivation of a gene may be carried out by any of the techniques well known to those skilled in the art. In particular, the inactivation of a gene may be obtained by the introduction of a point mutation in the gene, by the partial or total deletion of the coding sequences of the gene, or instead by modification of the promoter of the gene. These different genetic modifications will be carried out according to any one of the molecular biology techniques well known to those skilled in the art.

For example, attenuated viral strains of human metapneumoviruses have been obtained by deletion of the genes coding for the SH, G and/or M2-2 accessory proteins (see table 2).

The SH protein is a type II membrane protein, the functions of which are not yet completely characterised. In the case of hRSV, the deletion of the gene coding for the SH protein generates a recombinant virus capable of reproducing in vitro, the virulence of which is attenuated in the upper respiratory tractus of mice, but not in the lower part of said tractus (Bukreyev et al., 1997). In the case of hMPV, the functions of the SH protein are still under evaluation.

The G protein is also a type II membrane protein, its C-terminal end being outside of the cell. This protein is non-essential for the assembly of viral constituents, and for replication in vitro. For hRSV, it has been shown that the deletion of the gene coding this protein attenuated the virulence of the strain during infection of the respiratory tracts of mice (Teng et al., 2001). For hMPV, the functions of the G protein are still under evaluation.

According to an embodiment of the invention, the attenuated viral strain is characterised in that the genetic modifications comprise the inactivation of the two genes coding for the G protein and the SH protein.

According to another particular embodiment, the inactivation of the two genes corresponds to the complete deletion of one or the other or both genes coding for the G and SH proteins.

According to an embodiment of the invention, said attenuated strain has been genetically modified by introduction of at least one exogenous gene.

This exogenous gene could in particular be a gene coding for a viral antigen.

In the sense of the invention, “viral antigen” is taken to mean a protein element or element of another nature, expressed by a virus, that the immunological system of an individual recognises as foreign and which causes an immune response in said individual, notably the production of specific antibodies.

The viral antigens could in particular be selected from the antigens expressed by at least one influenza virus, or by at least one virus of the pneumovirus family, such as hRSV, or by at least one virus of the Paramyxoviridae family, such as the parainfluenza virus.

More particularly, said viral antigen could be chosen from all or part of the F protein of hRSV, and all or part of the haemagglutinin of influenza or parainfluenza viruses. According to another embodiment, said viral antigen is the F protein of hRSV, in its pre-fusion stabilised conformation as described in the article (Krarup A et al. 2015).

Such an attenuated viral strain comprising in addition an exogenous viral antigen will make it possible, during the administration thereof to a patient, to generate a multiple immune response, both against the expressed exogenous viral antigen and against hMPV. Such a strain making it possible to obtain a combined immune response against several viruses, further to a single administration, is highly advantageous.

Viral Strain of hMPV C-85473

According to a preferred embodiment of the invention, said attenuated strain is derived from a human metapneumovirus comprising the genome sequence represented by the sequence SEQ ID NO. 1.

Preferentially, said attenuated strain is derived from a viral strain of human metapneumovirus, designated C-85473, which was isolated from a patient sample in Canada. This strain belongs to the sub-group A1 of metapneumoviruses.

The complete genome sequence of this viral strain C-85473, comprising 13394 nucleotides, was disclosed for the first time in the patent application FR1856801. It is here represented in the list of sequences under the reference SEQ ID NO. 1.

The strain C-85473 is characterised by high fusogenic capacities, enabling it to penetrate into the target cells at a high frequency and/or a high degree of infection (Dubois et al., 2017). The high fusogenic capacity of this strain makes it possible to generate syncytia, i.e. giant multinucleated cells, particularly extended, constituted of a very large number of cell nuclei.

According to a preferred embodiment of the invention, the attenuated viral strain is derived from this strain C-85473 comprising the genome sequence represented by the sequence SEQ ID NO. 1.

The aim of the genetic modifications introduced into this strain C-85473 to obtain a so-called “derived” strain is to attenuate the virulence of said original strain, and not to modify the identity of its genome.

In particular, these genetic modifications only concern genes coding for proteins non-essential for the growth of the virus, otherwise called “accessory proteins”, such as SH and G proteins.

Advantageously, in this strain genetically modified in order to become “attenuated”, the peptide sequence of the F protein of the original strain rC-85473 is not modified, and thus has the same peptide sequence as the original strain.

In an entirely preferred manner, the attenuated viral strain is chosen from:
(i) A viral strain derived from the strain C-85473, genetically modified by inactivation of the gene coding for the SH protein;
(ii) A viral strain derived from the strain C-85473, genetically modified by inactivation of the gene coding for the G protein; and
(iii) A viral strain derived from the strain C-85473, genetically modified by inactivation of the gene coding for the SH protein and the gene coding for the G protein.

A viral strain illustrating embodiment (i) is in particular the strain used in the examples of the present application, comprising the nucleotide sequence such as represented in SEQ ID NO. 2.

A viral strain illustrating embodiment (ii) is in particular the strain used in the examples of the present application, comprising the nucleotide sequence such as represented in SEQ ID NO. 3.

According to an embodiment of the invention, the nucleotide sequence of the attenuated viral strain C-85473 could be, in addition, genetically modified by the introduction of at least one exogenous gene.

Thus, the attenuated viral strain C-85473 has a genome sequence that comprises at least one exogenous gene. This exogenous gene could in particular be a gene coding for a viral antigen.

Said viral antigen could in particular be selected from antigens expressed by at least one influenza virus, or by at least one virus of the Pneumoviridae family, such as hRSV, or by at least one virus of the Paramyxoviridae family, such as the parainfluenza virus.

More particularly, said viral antigen could be chosen from all or part of the F protein of hRSV, and all or part of the haemagglutinin of influenza or parainfluenza viruses.

According to another embodiment, said viral antigen is the F protein of hRSV, preferably in its stabilised pre-fusion conformation such as described in the article (Krarup A et al., 2015).

Other characteristics of these attenuated strains derived from the strain C-85473 comprising the genome sequence represented by the sequence SEQ ID NO. 1 are described in patent application FR1856801 and international application PCT/FR2019/051759.

Origin of the Attenuated Viral Strains

The attenuated viral strains used in the implementation of the present invention may be derived from various origins.

The attenuated viral strain could have been isolated in a patient suffering from a viral infection by pneumovirus, notably by a hMPV or a hRSV. Indeed, certain infectious viral strains may spontaneously have an attenuated character.

The attenuated viral strain may also have been genetically modified, from a non-attenuated viral strain.

According to a first embodiment, the attenuated viral strain could be obtained by reproduction of said virus on cells in culture. Frozen samples of infectious viral particles thus produced could notably be supplied by academic laboratories or hospitals.

According to a second embodiment, the attenuated viral strain could be obtained from DNA sequences using reverse genetics technology, notably described in the articles (Biacchesi et al., 2004) and (Aerts et al., 2015).

The principle of this technology, which enables the production of recombinant hMPVs, is based for example on the use of a hamster kidney cell line (BHK-21) modified to constitutively express the RNA polymerase of bacteriophage T7 (BHK-T7 or BSR-T7/5 cells).

The genomic elements are spread out in five plasmid elements: a plasmid coding the antigenome of hMPV and 4 “satellite” plasmids, coding for the viral proteins of the transcription machinery (L, P, N and M2-1).

After co-transfection of the hMPV antigenomic plasmid and four “satellite” plasmids in these cells, an RNA strand corresponding to the viral genomic strand (negative RNA strand), is transcribed by the T7 polymerase from its promoter.

The four proteins involved in the viral transcription of hMPV are in fact expressed by the transfected host cells to constitute an active RNA-dependant RNA polymerase (RdRP) complex. This functional viral polymerase thus transcribes genomic RNA into viral mRNA then replicates it into new molecules of viral genomic RNA, via the transcription of template strands.

The translation and the assembly of viral proteins with genomic RNA thus enable the budding of infectious hMPV particles from the cytoplasmic membrane of transfected BHK-T7 cells. Next, amplification of the recombinant viruses is enabled thanks to the addition to the co-culture of LLC-MK2 cells (ATCC CCL-7), permissive to infection.

Thus, from the sequences described in the present application, and appropriate host cells, those skilled in the art are capable of creating a functional recombinant enveloped virus comprising one of these sequences.

### Method for Producing a Viral Vaccine

According to another aspect, the present invention relates to a method for producing a viral vaccine such as defined above, comprising the following steps:
a) Infection of cells in culture of the ECACC 09070703 line by an attenuated viral strain derived from a human metapneumovirus;
b) Culture of said cells infected at step (a) for a duration comprised between 2 and 14 days in a suitable medium;
c) Harvesting of the viral vaccine constituted of infectious viral particles of said attenuated viral strain produced during step (b).

The attenuated viral strain used at step (a) will have been obtained notably by one of the technologies described above.

It is understood that this method could comprise additional steps, optional and not indicated here.

Furthermore, according to a particular embodiment, this method could consist exactly in the aforementioned three successive steps (a), (b) and (c).

Step (a) of infection will be carried out in appropriate conditions, such as for example in the following conditions:
the infection medium could be the OptiPRO™ SFM (Gibco™) culture medium suitable to the DuckCelt®-T17 cell line and complemented with 4 mM of L-Glutamine, 0.1% to 0.5% of Pluronic®, and trypsin of 0.1 μg/ml to 3 μg/ml of final volume. The trypsin is supplemented to the medium during infection but also every 2 to 3 days throughout the duration of viral production;
the cell density of the cells will be comprised between 0.5×10⁶ and 5×10⁶ cells/ml; and
the infection by the attenuated viral strain will be carried out at a multiplicity of infection (MOI) comprised between 1 and 0.0001.

Step (b) of culture of the infected cells will be carried out in appropriate conditions for normal growth of cells, well known to those skilled in the art. In particular:
the equipment for bio-producing cells in suspension could be of TubeSpin® type or a 50 ml to 200 ml flask on a Kühner type shaking platform incubator, or a 500 ml to 2 litre bioreactor of miniBioReactor Applikon BioTechnology type, or of UniVessel SU Sartorius Stedim Biotech type; and
the temperature of the culture medium will be comprised between 33 and 37° C.; the pH between 7 and 7.4; the stirring between 100 and 200 rpm and the oxygen content between 40 and 60%.

The cell culture step could last from 2 to 14 days, as a function of the cell growth and replicative capacities of the virus. The culture step could in particular be carried out for a duration of 3 to 12 days, or 4 to 10 days, or instead 5 to 9 days, or 6 to 8 days.

Step (c) of harvesting the infectious viral particles will be carried out by any technique well known to those skilled in the art, such as clarification of the production culture medium, followed by steps of purification, concentration and quantification of viral particles.

According to another aspect, the present invention relates to a viral vaccine capable of being obtained by the method described above. Said viral vaccine is constituted of infectious viral particles harvested at step (c) of the method described.

According to another embodiment, the present invention relates to a viral vaccine obtained by the method described above.

Pharmaceutical Composition

According to another aspect, the present invention relates to a pharmaceutical composition comprising said viral vaccine, and at least one pharmaceutically acceptable vehicle.

In the sense of the invention, “pharmaceutically acceptable vehicle” is taken to mean vehicles or excipients, that is to say “inactive” components, not having therapeutic properties. These vehicles or excipients may be administered to an individual or to an animal without significant deleterious effects or prohibitive adverse effects.

Other active components, normally used in pharmaceutical compositions, could be present in said composition: for example, adjuvants and/or excipients.

It is understood that the pharmaceutical composition according to the invention comprises at least one effective amount of the viral vaccine.

“Effective amount” is taken to mean, in the sense of the invention, an amount of attenuated virus strain sufficient to trigger an immune reaction in the organism to which it is administered.

The present pharmaceutical composition may also be designated as being a vaccinal composition.

The pharmaceutical compositions according to the present invention are notably suitable for administration orally, sublingually, or by inhalation.

According to a particular embodiment, the pharmaceutical composition according to the invention is suitable for administration by inhalation, that is to say nasally and/or orally.

Inhalation designates absorption by the respiratory tracts. It is in particular a method for absorption of compounds for therapeutic purposes, of certain substances in the form of gases, micro-droplets or powders in suspension.

Two types of administration by inhalation are distinguished:
administration by insufflation when the compositions are in the form of powders, and
administration by nebulisation when the compositions are in the form of aerosols (suspensions) or in the form of solutions, for example pressurised aqueous solutions. The use of a nebuliser or a spray will then be recommended for administering the pharmaceutical composition.

The galenic form of the pharmaceutical composition considered here is thus advantageously chosen from: a powder, an aqueous suspension of droplets or a pressurised solution.

According to a preferred embodiment, the pharmaceutical composition according to the invention is suitable for administration by nasal route, notably by inhalation.

Such a composition could be used as preventive vaccine, that is to say intended to stimulate a specific immune response before infection of an organism by a pathogenic virus.

Such a composition could also be used as therapeutic vaccine, that is to say intended to stimulate a specific immune response concomitantly with infection of an organism by a pathogenic virus.

Therapeutic Use

The present invention also pertains to the viral vaccine such as described above, or to the pharmaceutical composition such as described, for the use thereof as medicine, in other words for their therapeutic use.

Advantageously, this viral vaccine or this pharmaceutical composition will be used in therapy for the treatment and/or the prevention of viral infections.

The expression “treatment of viral infections” designates the fact of combatting an infection by a virus in an organism. The aim is to obtain a decrease in the infectious viral load (infectivity titre) in the organism, and preferably to obtain complete eradication of the virus from the organism. The term “treatment” also designates the action of attenuating the symptoms associated with the viral infection (respiratory syndrome, renal failure, fever, etc.).

The expression “prevention of viral infections” designates the fact of preventing, or at least decreasing the risk of onset of an infection in an organism. Thanks to this preventive action, the cells of said organism become less permissive to viral infection, and are thus more resistant to infection by said virus. In addition, the organism will have advantageously developed specific immune cells, making it possible to combat the aforementioned virus in a specific manner, thus limiting its entry into the cells of the organism.

More specifically, the invention relates to the viral vaccine or the pharmaceutical composition such as described above, for use in the prevention of viral infections, notably infections by pneumovirus, and notably by human metapneumovirus and/or human respiratory syncytial virus.

According to a first embodiment, said viral vaccine or said pharmaceutical composition is used in the prevention of infections by pneumoviruses.

According to a second embodiment, said viral vaccine or said pharmaceutical composition is used in the prevention of infections by a human metapneumovirus.

According to a third embodiment, said viral vaccine or said pharmaceutical composition is used in the prevention of infections by an orthopneumovirus, in particular by the human respiratory syncytial virus.

According to another aspect, the invention relates to the viral vaccine or the pharmaceutical composition such as described above, for use in the treatment of viral infections, notably infections by pneumoviruses, and more particularly by human metapneumovirus and/or human respiratory syncytial virus.

Indeed, said viral vaccine or pharmaceutical composition comprising it could be used, in certain cases and under certain conditions, in a therapeutic approach in individuals already infected by one of these viruses, notably in adult individuals.

The present invention also relates to a method for preventing viral infection in humans, notably an infection by pneumoviruses, more particularly by human metapneumovirus and/or by a human respiratory syncytial virus, comprising the administration to individuals liable to be infected by such a virus of an effective amount of a viral vaccine such as described above, or a pharmaceutical composition comprising it.

Said vaccine or said composition for the use thereof in the prevention and/or the treatment of viral infections are intended for all types of individuals, intended for any type of individual, not just the new born but also elderly adult individuals.

According to a preferred embodiment of the invention, said vaccine or said composition for the use thereof in the prevention and/or the treatment of viral infections are intended for paediatric use, that is to say are intended to be administered to a paediatric population.

In the sense of the invention, a paediatric population designates a population of individuals constituted of persons less than 18 years old, and more specifically young children less than 5 years old, and infants.

Indeed, viruses of the Pneumoviridae family mainly infect these individuals, who have a tendency to present a so-called “naïve” immunity, and thus less strong, than older individuals.

Finally, the present application also pertains to a kit for the implementation of the method for preparing a viral vaccine, comprising:
The immortalised cell line ECACC 09070703; and
An attenuated viral strain derived from a human metapneumovirus comprising the genome sequence represented by the sequence SEQ ID NO. 1.

Said attenuated viral strain could notably have been genetically modified, notably by inactivation of the gene coding for the SH protein and/or the gene coding for the G protein of said metapneumovirus strain.

In addition, said attenuated viral strain could have a genome sequence that comprises at least one exogenous gene. This exogenous gene could in particular be a gene coding for a viral antigen derived from another virus, such as for example all or part of the F protein of hRSV, and/or all or part of the haemagglutinin of influenza or parainfluenza viruses.

According to a preferred aspect, said viral strain will be in particular one of the strains described in the examples of the present application:
i) The viral strain comprising the nucleotide sequence such as represented in SEQ ID NO. 2, optionally comprising in addition at least one exogenous gene; or
ii) The viral strain comprising the nucleotide sequence such as represented in SEQ ID NO. 3, optionally comprising in addition at least one exogenous gene.

This kit could also include other elements, such as for example the culture medium suitable for the growth of the cell line, and/or directions for use specifying the ideal conditions for the preparation of the live attenuated viral vaccine.

### EXAMPLES

### Example 1. Use of the DuckCelt®-T17 Cell Line for the Replication of Wild Viral Strains of the Sub-Group A1 (C-85473 and CAN99-81), the Sub-Group B1 (CAN97-82) and the Sub-Group B2 (CAN98-75)

The DuckCelt®-T17 cells are cultured in OptiPro+L-glutamine 4 mM final medium in TubeSpin 50 ml in a Kühner shaker at the speed of 175 rpm, at 37° C. with 5% CO2 and 85% hygrometry, and diluted to 1×10⁶ cells/ml in 10 ml of medium. They were infected with a multiplicity of infection (MOI) of 0.01 by wild viral strains (non GFP) of the sub-group A1 (C-85473 and CAN99-81), the sub-group B1 (CAN97-82) and the sub-group B2 (CAN98-75) in the presence of trypsin (T6763 Sigma) at 0.5 μg/ml.

The cells are counted in trypan blue every 2 to 3 days to evaluate cell growth as well as cell death during viral infection. The results are shown in FIG. 1a.

Viral production is measured by assaying the number of infectious particles per ml in the culture medium (expressed in TCID₅₀/ml) from samples taken every 2 to 3 days up to 14 days post-infection. The results are shown FIG. 1b.

The viral replication kinetics are stopped when cell death reaches more than 50%, i.e. at 8 days post-infection for viral strain C-85473 and at 14 days post-infection for viral strains CAN99-81, CAN97-82 and CAN98-75.

The results show that only the virus C-85473 amplifies during the infection of DuckCelt®-T17 cells with an increase in the viral load by more than 2 log 10 in 7 days. The virus CAN98-75 demonstrates low viral production from 4 to 9 days post-infection whereas the viruses CAN99-81 and CAN97-82 are detectable at levels less than the initial inoculum or below the detection thresholds up to 14 days post-infection.

In conclusion, the viral strain C-85473 has a replication capacity on DuckCelt®-T17 cells very significantly higher than those of other hMPV viral strains. In particular, the level of cell death observed as of 8 days post-infection indicates that the infection capacities of this strain on this cell line are significant.

### Example 2. Use of the DuckCelt®-T17 Cell Line for the Replication of the Wild Strain C-85473 WT, Recombinant Because Expressing Green Fluorescent Protein (GFP), and the Recombinant Viral Strains ΔSH-C-85473 (GFP) and ΔG-C-85473 (GFP)

The viral strains used in this example have the following genome sequences:
The sequence C-85473 WT-GFP is represented in SEQ ID NO. 4;
The sequence ΔSH-C-85473-GFP is represented in SEQ ID NO. 5;
The sequence ΔG-C-85473-GFP is represented in SEQ ID NO. 6.

The DuckCelt®-T17 cells are maintained in culture in OptiPro+L-glutamine 4 mM final medium in TubeSpin 50 ml in a Kühner shaker at the speed of 175 rpm, at 37° C. with 5% CO2 and 85% hygrometry.

Before infection, the cells are diluted to 1×10⁶ cells/ml in 10 ml of culture medium then are infected by the wild recombinant hMPVs (C-85473 WT), or deleted of the genome sequence coding the SH protein (ΔSH-C-85473) or deleted of the genome sequence coding the G protein (ΔG-C-85473), at a multiplicity of infection (MOI) of 0.01 in the presence of trypsin (T6763 Sigma) at 0.5 μg/ml. The “mock” cells are cells that have not been infected and constitute the negative control of the experiment.

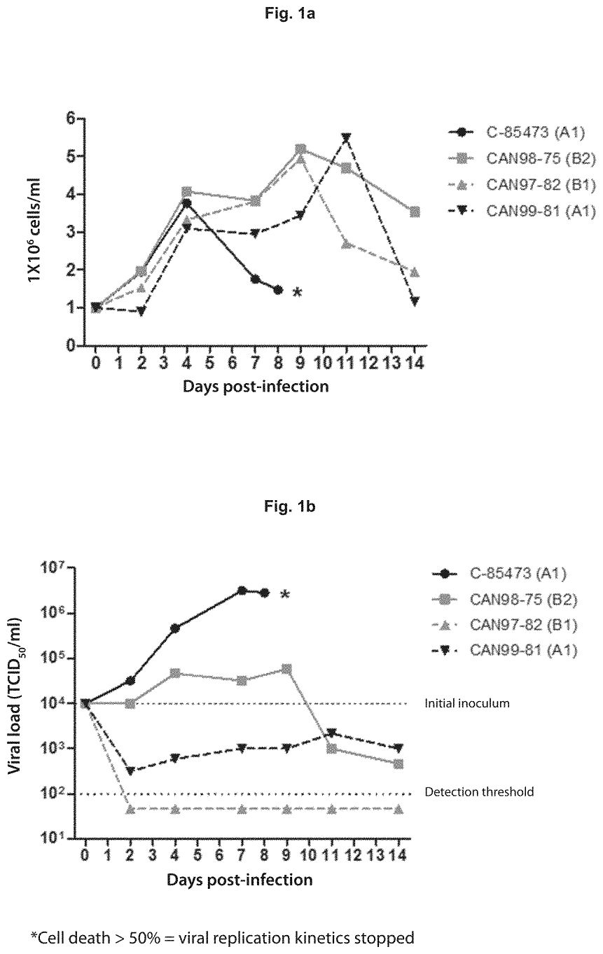

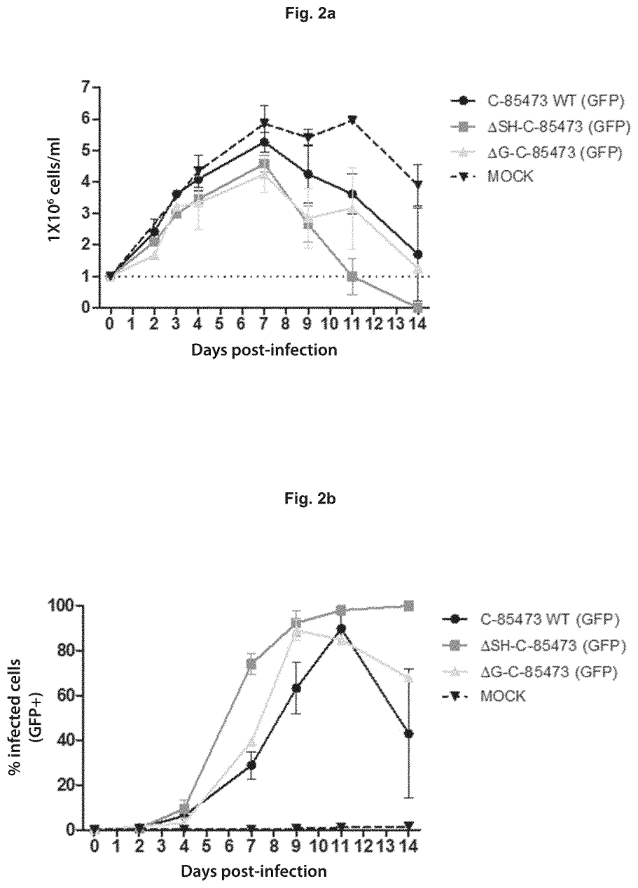

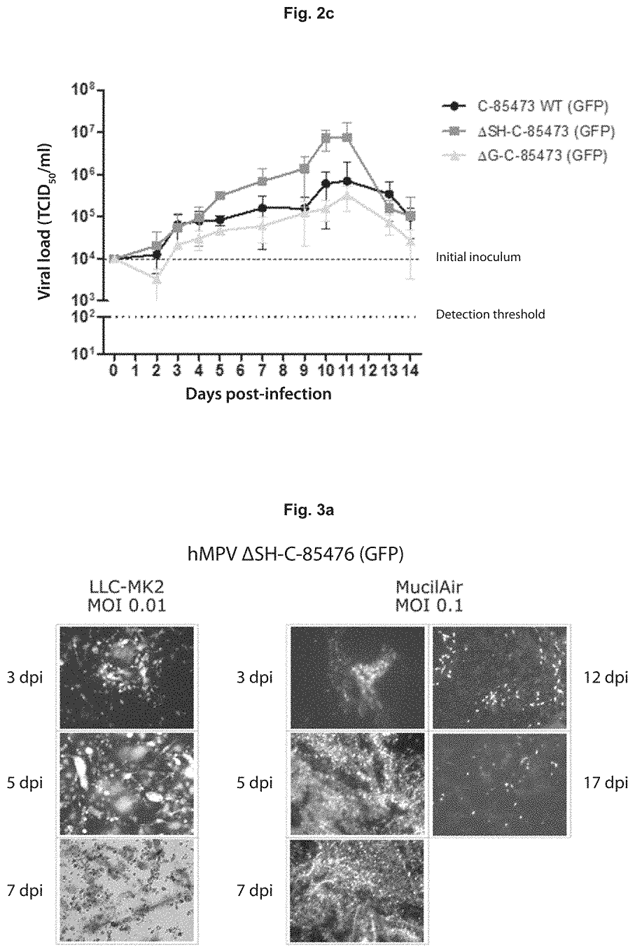

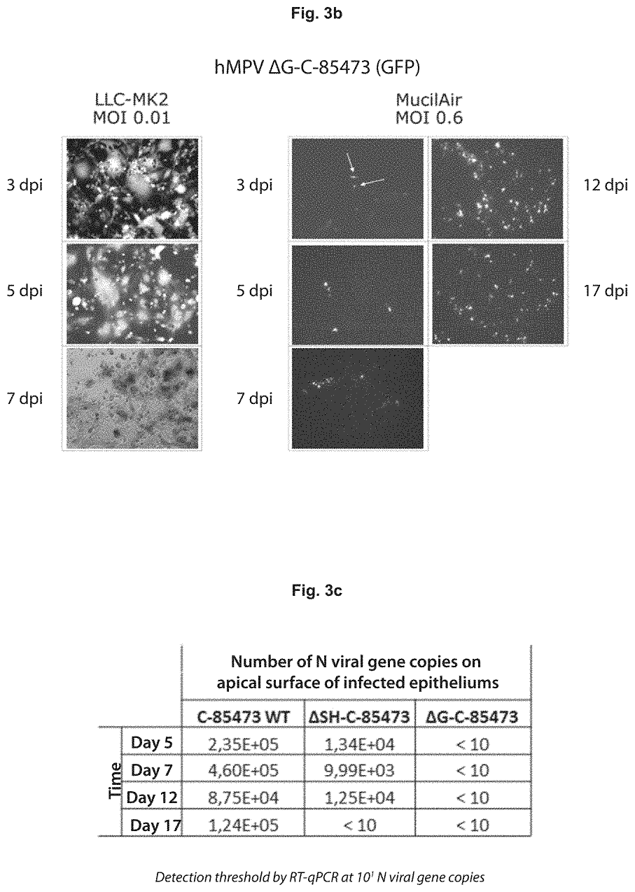

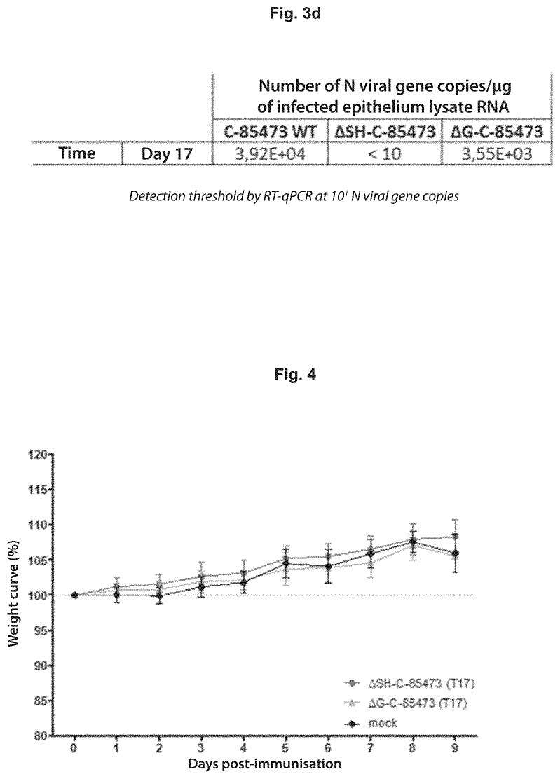

The cells are counted in trypan blue every 2 to 3 days after infection to evaluate cell growth in the course of infection. The results obtained are shown FIG. 2a.

The viral replication kinetics are stopped when cell death reaches more than 50%, i.e. after 14 days on average.

The percentage infected cells is evaluated by flow cytometry (detection of the expression of GFP expressed by the recombinant viruses) during the 14 days of viral kinetics. The results obtained are shown in FIG. 2b.

Viral production is measured by assaying the number of infectious particles per ml of culture medium (expressed in TCID₅₀/ml) from samples taken every 2 to 3 days during the 14 days of kinetics. The results obtained are shown in FIG. 2C.

The results obtained represent four independent experiments.

These results show that, in these culture and viral infection conditions, the DuckCelt®-T17 cells (i) reach their maximum concentration at 7 days after infection; (ii) are infected by the wild viral strain and the modified viral strains ΔSH-C-85473 and ΔG-C-85473 to more than 80%, between 9 and 11 days post-infection.

The viral production peak for the three viral strains is situated at 11 days post-infection.

In conclusion, the results indicate that the DuckCelt®-T17 line is “permissive”, that it can be infected by the recombinant viruses C-85473 and in particular the live attenuated viruses ΔSH-C-85473 and ΔG-C-85473, and enable the production of viral particles.

### Example 3. Characterisation of the Viral Particles Obtained According to the Culture Method of Example 2

This example relates to the measurement of the replicative capacities of the recombinant viruses ΔSH-C-85473 and ΔG-C-85473 produced on DuckCelt®-T17 cells, in comparison with a recombinant virus C-85473 WT:
(i) in monkey kidney epithelial cells LLC-MK2 (ATCC-CCL7) and
(ii) in a 3D reconstituted human pulmonary epithelium model (MucilAir™, Epithelix) and cultured at the air-liquid interface.

A cell lawn of LLC-MK2 cells and MucilAir™ healthy reconstituted human respiratory epitheliums were infected by:
recombinant hMPV ΔSH-C-85473 at MOI (multiplicity of infection) of 0.01 and 0.1, respectively;
recombinant hMPV ΔG-C-85473 at MOI (multiplicity of infection) of 0.01 and 0.65, respectively.

Photos of the infected cells taken at 3, 5, 7, 12 and 17 days post-infection are shown in FIGS. 3a (ΔSH-C-85473) and 3b (ΔG-C-85473).

From 5 days of infection, viral replication within infected epitheliums and viral secretion at their apical pole was evaluated by RT-qPCR (number of copies of the N viral gene) from epithelial lysates and apical surface washings, respectively.

The results are shown in FIGS. 3c (surface washings) and 3d (epithelial lysates).

The results obtained indicate that the live attenuated viruses ΔSH-C-85473 and ΔG-C-85473 produced on DuckCelt®-T17 cells are functional and conserve their capacity to infect LLC-MK2 cells and in particular the reconstituted 3D human pulmonary epithelium model (MucilAir™, Epithelix).

It is to be noted that the attenuated strains ΔSH-C-85473 and ΔG-C-85473 replicate little, and in a less sustained manner over time, in the human epithelium model compared to a wild virus C-85473, as is shown in FIG. 3d; whereas their rate of replication on DuckCelt®-T17 (and accessorily LLC-MK2) cells is very satisfactory.

These two characteristics make them excellent vaccine candidates, these attenuated viral strains being able to be produced easily in vitro but only having a very moderate risk when introduced into a living organism, with regard to the results obtained in this human infection model physiologically close to the conditions of an organism.

### Example 4. The Recombinant Viruses ΔSH-C-85473 and ΔG-C-85473 Produced on DuckCelt®-T17 Cells Conserve their Attenuated Character In Vivo

BALB/c mice 4 to 6 weeks old were infected by intranasal route with:
5×10⁵ TCID₅₀ of the vaccine candidates ΔSH-C-85473 or ΔG-C-85473 produced on DuckCelt®-T17 cells and concentrated by ultracentrifugation, or
Optimem culture medium (mock) as control (10 mice per group).

Daily monitoring of the weight and the mortality of the mice was carried out for 9 days after infection. The results are shown in FIG. 4. The results obtained indicate that mice infected with the live attenuated viruses ΔSH-C-85473 or ΔG-C-85473 produced on DuckCelt®-T17 cells exhibit no signs of pathology or mortality, thus demonstrating the attenuating character of these viruses produced on DuckCelt®-T17 cells.

TABLE 4
Summary of sequences presented in the sequence listing

| | Description |
|---|---|
| SEQ ID NO. 1 | Whole genome sequence of wild-type strain hMPV C-85473 |
| SEQ ID NO. 2 | Whole sequence of recombinant virus hMPV ΔSH C-85473 |
| SEQ ID NO. 3 | Whole sequence of recombinant virus hMPV ΔG C-85473 |
| SEQ ID NO. 4 | Whole genome sequence of wild-type strain hMPV 0-85473 |
| | combined with GFP sequence (inserted between |
| | nucleotide 40 and 784) |
| SEQ ID NO. 5 | Whole genome sequence of wild-type strain hMPV C-85473, |
| | combined with GFP sequence (inserted between |
| | nucleotide 40 and 784) |
| SEQ ID NO. 6 | Whole genome sequence of wild-type strain hMPV C-85473, |
| | combined with GFP sequence (inserted between |
| | nucleotide 40 and 784) |

### PATENT REFERENCES

WO 2007/077256
WO 2009/004016
WO 2012/001075
WO 2005/014626
U.S. Pat. No. 8,841,433
FR1856801

### SCIENTIFIC LITERATURE

### Bibliographic References by Order of Citation in the Text

Mazur N I, Higgins D, Nunes M C, Melero J A, Langedijk A C, Horsley N, Buchholz U J, Openshaw P J, McLellan J S, Englund J A, Mejias A, Karron R A, Simoes E A, Knezevic I, Ramilo O, Piedra P A, Chu H Y, Falsey A R, Nair H, Kragten-Tabatabaie L, Greenough A, Baraldi E, Papadopoulos N G, Vekemans J, Polack F P, Powell M, Satav A, Walsh E E, Stein R T, Graham B S, Bont L J; Respiratory Syncytial Virus Network (ReSViNET) Foundation. The respiratory syncytial virus vaccine landscape: lessons from the graveyard and promising candidates. Lancet Infect Dis. 2018 Jun. 15.
Jordan I, Vos A, Beilfuss S, Neubert A, Breul S, Sandig V. An avian cell line designed for production of highly attenuated viruses. Vaccine. 2009 Jan. 29; 27(5):748-56.
Petiot E, Proust A, Traversier A, Durous L, Dappozze F, Gras M, Guillard C, Balloul J M, Rosa-Calatrava M. Influenza viruses production: Evaluation of a novel avian cell line DuckCelt®-T17. Vaccine. 2018 May 24; 36(22):3101-3111.
Huck B, Scharf G, Neumann-Haefelin D, Puppe W, Weigl J, Falcone V. Novel human metapneumovirus sublineage. Emerg Infect Dis. 2006 January; 12(1):147-50.
Nidaira M, Taira K, Hamabata H, Kawaki T, Gushi K, Mahoe Y, Maeshiro N, Azama Y, Okano S, Kyan H, Kudaka J, Tsukagoshi H, Noda M, Kimura H. Molecular epidemiology of human metapneumovirus from 2009 to 2011 in Okinawa, Japan. Jpn J Infect Dis. 2012 July; 65(4):337-40.
Herfst S, de Graaf M, Schrauwen E J, Sprong L, Hussain K, van den Hoogen B G, Osterhaus A D, Fouchier R A. Generation of temperature-sensitive human metapneumovirus strains that provide protective immunity in hamsters. J Gen Virol. 2008 JuI; 89(Pt 7):1553-62.
Wei Y, Zhang Y, Cai H, Mirza A M, Iorio R M, Peeples M E, Niewiesk S, Li J. Roles of the putative integrin-binding motif of the human metapneumovirus fusion (f) protein in cell-cell fusion, viral infectivity, and pathogenesis. J Virol. 2014 April; 88(8):4338-52.
Yu C M, Li R P, Chen X, Liu P, Zhao X D. Replication and pathogenicity of attenuated human metapneumovirus F mutants in severe combined immunodeficiency mice. Vaccine. 2012 Jan. 5; 30(2):231-6.
Liu P, Shu Z, Qin X, Dou Y, Zhao Y, Zhao X. A live attenuated human metapneumovirus vaccine strain provides complete protection against homologous viral infection and cross-protection against heterologous viral infection in BALB/c mice. Clin Vaccine Immunol. 2013 August; 20(8): 1246-54.
Zhang Y, Wei Y, Zhang X, Cai H, Niewiesk S, Li J. Rational design of human metapneumovirus live attenuated vaccine candidates by inhibiting viral mRNA cap methyltransferase. J Virol. 2014 October; 88(19):11411-29
Biacchesi S, Skiadopoulos M H, Tran K C, Murphy B R, Collins P L, Buchholz U J. Recovery of human metapneumovirus from cDNA: optimization of growth in vitro and expression of additional genes. Virology. 2004; 321(2):247-59
Biacchesi S, Pham Q N, Skiadopoulos M H, Murphy B R, Collins P L, Buchholz U J. Infection of nonhuman primates with recombinant human metapneumovirus lacking the S H, G, or M2-2 protein categorizes each as a nonessential accessory protein and identifies vaccine candidates. Journal of virology. 2005; 79(19):12608-13.
Buchholz U J, Biacchesi S, Pham Q N, Tran K C, Yang L, Luongo C L, Skiadopoulos M H, Murphy B R, Collins P L. Deletion of M2 gene open reading frames 1 and 2 of human metapneumovirus: effects on RNA synthesis, attenuation, and immunogenicity. J Virol. 2005 June; 79(11):6588-97.
Schickli J H, Kaur J, Macphail M, Guzzetta J M, Spaete R R, Tang R S. Deletion of human metapneumovirus M2-2 increases mutation frequency and attenuates growth in hamsters. Virol J. 2008 Jun. 3; 5:69.
Pham Q N, Biacchesi S, Skiadopoulos M H, Murphy B R, Collins P L, Buchholz U J. Chimeric recombinant human metapneumoviruses with the nucleoprotein or phosphoprotein open reading frame replaced by that of avian metapneumovirus exhibit improved growth in vitro and attenuation in vivo. J Virol. 2005 December; 79(24):15114-22.
Karron R A, San Mateo J, Wanionek K, Collins P L, Buchholz U J. Evaluation of a Live Attenuated Human Metapneumovirus Vaccine in Adults and Children. J Pediatric. Infect Dis Soc. 2018 Feb. 19; 7(1):86-89
Tang R S, Schickli J H, MacPhail M, Fernandes F, Bicha L, Spaete J, Fouchier R A, Osterhaus A D, Spaete R, Haller A A. Effects of human metapneumovirus and respiratory syncytial virus antigen insertion in two 3′ proximal genome positions of bovine/human parainfluenza virus type 3 on virus replication and immunogenicity. J Virol. 2003 October; 77(20):10819-28.
Tang R S, Mahmood K, Macphail M, Guzzetta J M, Haller A A, Liu H, Kaur J, Lawlor H A, Stillman E A, Schickli J H, Fouchier R A, Osterhaus A D, Spaete R R. A host-range restricted parainfluenza virus type 3 (PIV3) expressing the human metapneumovirus(hMPV) fusion protein elicits protective immunity in African green monkeys. Vaccine. 2005 Feb. 25; 23(14):1657-67.
Russell C J, Jones B G, Sealy R E, Surman S L, Mason J N, Hayden R T, Tripp R A, Takimoto T, Hurwitz J L. A Sendai virus recombinant vaccine expressing a gene for truncated human metapneumovirus (hMPV) fusion protein protects cotton rats from hMPV challenge. Virology. 2017 September; 509:60-66.
Bukreyev A, Whitehead S S, Murphy B R, Collins P L. Recombinant respiratory syncytial virus from which the entire S H gene has been deleted grows efficiently in cell culture and exhibits site-specific attenuation in the respiratory tract of the mouse. J Virol. 1997 December; 71(12):8973-82.
Teng M N, Whitehead S S, Collins P L. Contribution of the respiratory syncytial virus G glycoprotein and its secreted and membrane-bound forms to virus replication in vitro and in vivo. Virology. 2001 Oct. 25; 289(2):283-96.
Dubois J, Cavanagh M H, Terrier O, Hamelin M E, Lina B, Shi R, et al. Mutations in the fusion protein heptad repeat domains of human metapneumovirus impact on the formation of syncytia. The Journal of general virology. 2017; 98(6):1174-80.
Krarup A, Truan D, Furmanova-Hollenstein P, Bogaert L, Bouchier P, Bisschop I J, Widjojoatmodjo M N, Zahn R, Schuitemaker H, McLellan J S, Langedijk J P. A highly stable prefusion RSV F vaccine derived from structural analysis of the fusion mechanism. Nat Commun. 2015 Sep. 3; 6:8143.
Aerts L, Rhéaume C, Carbonneau J, Lavigne S, Couture C, Hamelin M E, Boivin G. Adjuvant effect of the human metapneumovirus (HMPV) matrix protein in HMPV subunit vaccines. J Gen Virol. 2015 April; 96(Pt 4):767-74.
Aerts L, Cavanagh M H, Dubois J, Carbonneau J, Rheaume C, Lavigne S, et al. Effect of in vitro syncytium formation on the severity of human metapneumovirus disease in a murine model. PloS one. 2015; 10(3):e0120283.

1-12. (canceled)

13. Method for producing a viral vaccine constituted of an attenuated viral strain derived from a human metapneumovirus, comprising steps of infection with said strain and culture of the immortalised cell line ECACC 09070703, filed on 7 Jul. 2009 at the European Collection of Authenticated Cell Cultures (ECACC, Salisbury, UK) under the number 09070703.

14. Method according to claim 13, characterised in that said attenuated strain has been genetically modified by inactivation of the gene coding for the SH protein and/or the gene coding for the G protein of metapneumovirus.

15. Method according to claim 13, characterised in that said attenuated strain has been genetically modified by introduction of at least one exogenous gene.

16. Method according to one of claim 13, characterised in that said attenuated strain is derived from a human metapneumovirus comprising the genome sequence represented by the sequence SEQ ID NO. 1.

17. Method according to claim 13, comprising the following steps:
a) Infection of cells in culture of the line ECACC 09070703 by said attenuated viral strain derived from a human metapneumovirus;
b) Culture of said cells infected at step (a) for a duration comprised between 2 and 14 days in a suitable medium;
c) Harvesting of the viral vaccine constituted of infectious viral particles of said attenuated viral strain produced during step (b).

18. Viral vaccine obtained by the method according to claim 17.

19. Pharmaceutical composition comprising the viral vaccine according to claim 18, and at least one pharmaceutically acceptable vehicle.

20. Pharmaceutical composition according to claim 19, suitable for administration by nasal route.

21. Method for preventing viral infection in humans, comprising the administration to individuals liable to be infected by such a virus of an effective amount of a viral vaccine according to claim 18.

22. Method according to claim 21, characterised in that said individuals are persons less than 18 years old.

23. Kit for the implementation of the method according to claim 17, comprising:
the immortalised cell line ECACC 09070703; and
an attenuated viral strain derived from a human metapneumovirus comprising the genome sequence represented by the sequence SEQ ID NO. 1.

24. Method for preventing viral infection in humans, comprising the administration to individuals liable to be infected by such a virus of an effective amount of a pharmaceutical composition according to claim 19.

25. Method according to claim 21, wherein the virus is a pneumovirus.

26. Method according to claim 25, wherein the pneumovirus is a human metapneumovirus (hMPV).

27. Method according to claim 21, wherein the virus is a human respiratory syncytial virus (hRSV).

28. Method according to claim 24, wherein the virus is a pneumovirus.

29. Method according to claim 28, wherein the pneumovirus is a human metapneumovirus (hMPV).

30. Method according to claim 24, wherein the virus is a human respiratory syncytial virus (hRSV).
